(19) United States
(12) Patent Application Publication
(10) Pub. No.: US 2012/0104940 A1
(43) Pub. Date: May 3, 2012

(54) NOVEL COMPOUNDS FOR ORGANIC ELECTRONIC MATERIAL AND ORGANIC ELECTRONIC DEVICE USING THE SAME

(75) Inventors: Hyo-Nim Shin, Seoul (KR); Chi Sik Kim, Seoul (KR); Young Jun Cho, Seoul (KR); Hyuck Joo Kwon, Seoul (KR); Bong Ok Kim, Seoul (KR); Sung Min Kim, Seoul (KR); Seung Soo Yoon, Seoul (KR)

(73) Assignee: Rohm and Haas Electronic Materials Korea Ltd., Chungcheon-do (KR)

(21) Appl. No.: 13/262,359

(22) PCT Filed: Mar. 19, 2010
(86) PCT No.: PCT/KR2010/001708

§ 371 Date: Jan. 3, 2012

(30) Foreign Application Priority Data
Mar. 31, 2009 (KR) 10-2009-0027221

(51) Int. Cl. H05B 33/14; C07C 13/62; C07C 211/60; C07F 7/10; C07D 487/14

(52) U.S. Cl. 313/504; 556/413; 548/418; 544/212; 564/429; 546/276.7; 544/331; 585/27

(57) ABSTRACT

The present invention relates to novel compounds for organic electronic material, and organic electronic devices and organic solar cells using the same. The compounds for organic electronic material may be included in a hole transport layer, electron transport layer or hole injection layer, or may be used as host or dopant. With good luminous efficiency and excellent life property of the material, they may be used to manufacture OLEDs having very good operation life.

### FIELD OF THE INVENTION

The present invention relates to a novel compound for organic electronic material and an organic electronic device including the same. The compound for organic electronic material according to the present invention may be included in a hole transport layer, electron transport layer or hole injection layer, or may be used as host or dopant.

### BACKGROUND OF THE INVENTION

Among display devices, electroluminescent (EL) devices are advantageous in that they provide wide view angle, superior contrast and fast response rate as self-emissive display devices. In 1987, Eastman Kodak first developed an organic EL device using low-molecular-weight aromatic diamine and aluminum complex as a substance for forming an electroluminescent layer [Appl. Phys. Lett. 51, 913, 1987].

In an organic EL device, when a charge is applied to an organic layer formed between an electron injection electrode (cathode) and a hole injection electrode (anode), an electron and a hole are paired and emit light as the electron-hole pair is extinguished. The organic EL device is advantageous in that it can be formed on a flexible transparent substrate such as plastic, is operable with relatively low voltage (10 V or lower) as compared to plasma display panels or inorganic EL displays, consumes less power and provides excellent color. In an organic EL device, the most important factor that determines its performance including luminous efficiency and operation life is the electroluminescent material. Some requirements of the electroluminescent material include high electroluminescence quantum yield in solid state, high electron and hole mobility, resistance to decomposition during vacuum deposition, ability to form uniform film and stability.

Organic electroluminescent materials may be roughly classified into high-molecular-weight materials and low-molecular-weight materials. The low-molecular-weight materials may be classified into metal complexes and metal-free pure organic electroluminescent materials, depending on molecular structure. Chelate complexes such as tris(8-quinolato)aluminum, coumarin derivatives, tetraphenylbutadiene derivatives, bisstyrylarylene derivatives, oxadiazole derivatives, or the like are known. It is reported that electroluminescence from blue to red light in the visible region can be obtained using these materials. In order to realize a full-color OLED display, three electroluminescent materials of red, green and blue (RGB) are employed. Thus, development of RGB electroluminescent materials having high efficiency and long operation life is important in enhancing the properties of an organic EL device. In functional aspect, the electroluminescent materials may be divided into host materials and dopant materials. In general, an electroluminescent layer prepared by doping a dopant in a host is known to provide superior EL property. Recently, development of an organic EL device having high efficiency and long operation life is becoming an imminent task. Especially, considering the level of EL performance required for medium-to-large sized OLED panels, development of materials which are much superior to existing electroluminescent materials is urgently needed.

For blue electroluminescent materials, a lot of materials have been commercialized following Idemitsu Kosan's DPVBi (Compound d). In addition to the Idemitsu Kosan's blue material system, Kodak's dinaphthylanthracene (Compound e) and tetra(t-butyl)perylene (Compound f) are known, but more researches and developments are necessary. Until now, Idemitsu Kosan's distyryl compound system is known to have the best efficiency. It exhibits a power efficiency of 6 lm/W and an operation life of 30,000 hours or longer. However, its sky-blue color is not appropriate for a full-color display is only thousands of hours. In general, blue electroluminescence becomes advantageous in terms of luminous efficiency if the electroluminescence wavelength is shifted a little toward a longer wavelength. But, then, it is not applicable to high-quality displays because pure blue color is not attained. Therefore, researches and developments to improve color purity, efficiency and thermal stability are highly required.

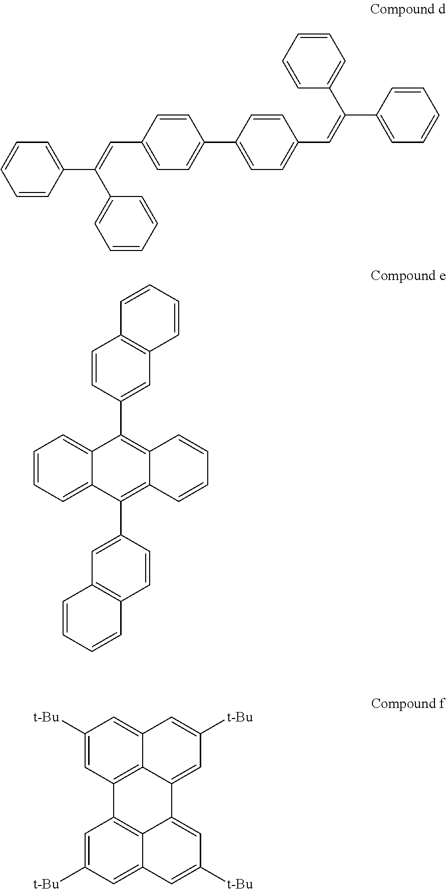

The hole injection/transport material may include copper phthalocyanine (CuPc), 4,4′-bis[N-(1-naphthyl)-N-phenylamino]biphenyl (NPB), N,N′-diphenyl-N,N′-bis(3-methylphenyl)-(1,1′-biphenyl)-4,4′-di amine (TPD), 4,4′,4″-tris(3-methylphenylphenylamino)triphenylamine (MTDATA), or the like. A device using these materials in the hole injection or transfer layer is problematic in efficiency and operation life. It is because, when an organic EL device is driven under high current, thermal stress occurs between an anode and the hole injection layer. The thermal stress significantly reduces the operation life of the device. Further, since the organic material used in the hole injection layer has very high hole mobility, the hole-electron charge balance may be broken and quantum yield (cd/A) may decrease.

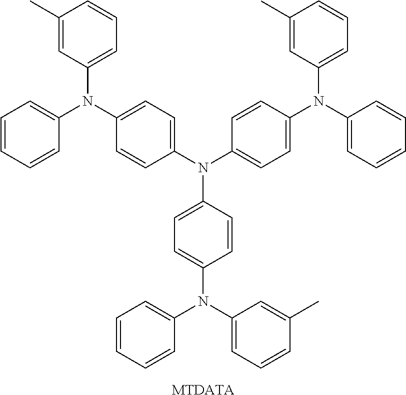

It is known that an amorphous compound providing good stability of thin film improves the operation life of the organic EL device. Glass transition temperature (T_{g}) may be a measure of the amorphousness. MTDATA has a glass transition temperature of 76° C. and cannot be said to have high amorphousness. These materials are not satisfactory in the operation life of the organic EL device, as well as in the luminous efficiency, which is determined by the hole injection/transport properties.

Representative examples of existing electron transport materials include aluminum complexes such as tris(8-hydroxyquinoline)aluminum(III) (Alq), which has been used prior to the multilayer thin film OLEDs disclosed in 1987 by Kodak, and beryllium complexes such as bis(10-hydroxybenzo-[h]quinolinato)beryllium (Bebq), which was reported in Japan in the middle of 1990s [T. Sato et al. J. Mater. Chem. 10 (2000) 1151]. However, as OLEDs have been commercialized since 2002, limitations of these materials have come to the fore. Thereafter, a lot of electron transport materials of high performance have been investigated and reported near to the level of commercialization.

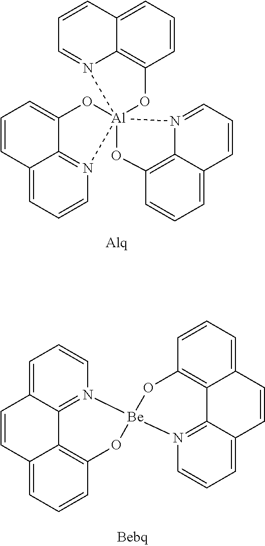

Non-metal electron transport materials having good features which have been reported up to the present include spiro-PBD [N. Johansson et al. Adv. Mater. 10 (1998) 1136], PyPySPyPy [M. Uchida et al. Chem. Mater. 13 (2001) 2680] and Kodak's TPBI [Y.-T. Tao et al. Appl. Phys. Lett. 77 (2000) 1575]. However, there remain various needs for improvement in terms of electroluminescent properties and lifetime.

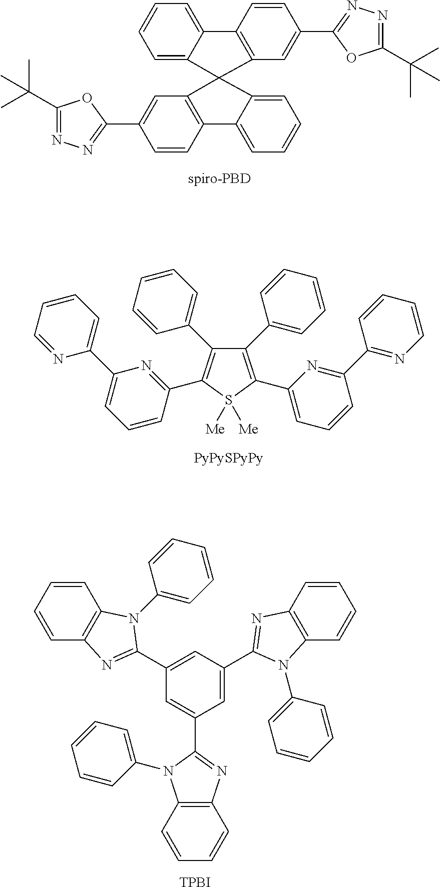

Particularly, it is to be noted that the existing electron transport materials have only slightly improved driving voltage or show the problem of markedly decreased operation life of the device. In addition, the materials exhibitive adverse effects such as deviation in device operation life for each color and deterioration of thermal stability. Due to these problems, it is difficult to achieve reasonable power consumption, increased luminance, etc. which are required in manufacturing large-sized OLED panels.

Up to the present, 4,4′-N,N′-dicarbazolebiphenyl (CBP) is the best known host material of a phosphorescent light-emitting material, and OLEDs with high efficiency including a hole blocking layer of BCP or BAlq are known. Also, high-performance OLEDs using BAlq derivatives as the host have been developed by Pioneer (Japan) or the like.

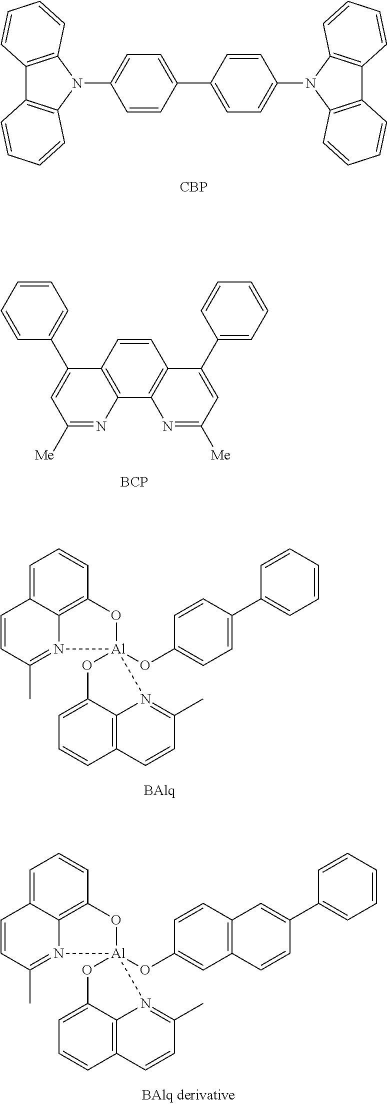

Although these materials are advantageous in view of light-emitting properties, their properties may be modified during high-temperature deposition process in vacuum because of low glass transition temperature and very poor thermal stability. The power efficiency of an OLED is determined by “power efficiency=(π/voltage)×current efficiency”. That is, the power efficiency is inversely proportional to the voltage, and the power efficiency should be improved to reduce power consumption of the OLED. In practice, an OLED employing a phosphorescent electroluminescent material exhibits fairly higher current efficiency (cd/A) than one employing a fluorescent electroluminescent material. However, use of BAlq or CBP as host of the phosphorescent electroluminescent material does not provide significant advantage over an OLED employing a fluorescent material in terms of power efficiency (lm/w), because of higher driving voltage. Furthermore, the result is not satisfactory in view of operation life of the OLED device. Accordingly, development of a host material capable of providing better stability and performance is still required.

### Technical Problem

Accordingly, an object of the present invention is to provide a compound for organic electronic material having luminous efficiency and device operation life improved over existing host or dopant materials and having superior backbone with appropriate color coordinates in order to solve the aforesaid problems. Another object of the present invention is to provide an organic electronic device employing the novel compound for organic electronic material in a hole injection layer, a hole transport layer, an electron transport layer or an electroluminescent layer.

### Technical Solution

The present invention provides a compound for organic electronic material represented by Chemical Formula 1 and an organic electronic device including the same. The compound for organic electronic material according to the present invention may be included in a hole injection layer, a hole transport layer or an electron transport layer, and may be used as a host or a dopant. With superior luminous efficiency and excellent life property, it may be used to manufacture an OLED device having very superior operation life.

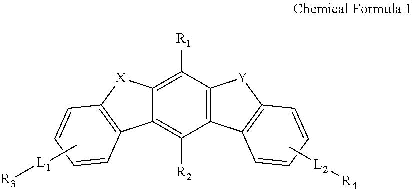

wherein

X and Y independently represent —C(R₅₁)(R₅₂)—, —N(R₅₃)—, —S—, —O—, —Si(R₅₄)(R₅₅)—, —P(R₅₆)—, —P(═O)(R₅₇)—, —C(═O)— or —B(R₅₈)—;

R₁ through R₄ and R₅₁ through R₅₈ independently represent hydrogen, deuterium, halogen, (C1-C30)alkyl with or without substituent(s), (C6-C30)aryl with or without substituent(s), (C6-C30)aryl with or without substituent(s) fused with one or more (C3-C30)cycloalkyl(s) with or without substituent(s), (C3-C30)heteroaryl with or without substituent(s), 5- to 7-membered heterocycloalkyl with or without substituent(s), 5- to 7-membered heterocycloalkyl fused with one or more aromatic ring(s) with or without substituent(s), (C3-C30)cycloalkyl with or without substituent(s), (C3-C30)cycloalkyl fused with one or more aromatic ring(s) with or without substituent(s), adamantyl with or without substituent(s), (C7-C30)bicycloalkyl with or without substituent(s), cyano, NR₂₁R₂₂, BR₂₃R₂₄, PR₂₅R₂₆, P(═O)R₂₂R₂₈ [wherein R₂₁ through R₂₈ independently represent (C1-C30)alkyl with or without substituent(s), (C6-C30)aryl with or without substituent(s), or (C3-C30)heteroaryl with or without substituent(s).], tri(C1-C30)alkylsilyl with or without substituent(s), di(C1-C30)alkyl(C6-C30)arylsilyl with or without substituent(s), tri(C6-C30)arylsilyl with or without substituent(s), (C6-C30)ar(C1-C30)alkyl with or without substituent(s), (C1-C30)alkyloxy with or without substituent(s), (C1-C30)alkylthio with or without substituent(s), (C6-C30)aryloxy with or without substituent(s), (C6-C30)arylthio with or without substituent(s), (C1-C30)alkoxycarbonyl with or without substituent(s), (C1-C30)alkylcarbonyl with or without substituent(s), (C6-C30)arylcarbonyl with or without substituent(s), (C2-C30)alkenyl with or without substituent(s), (C2-C30)alkynyl with or without substituent(s), (C6-C30)aryloxycarbonyl with or without substituent(s), (C1-C30)alkoxycarbonyloxy with or without substituent(s), (C1-C30)alkylcarbonyloxy with or without substituent(s), (C6-C30)arylcarbonyloxy with or without substituent(s), (C6-C30)aryloxycarbonyloxy with or without substituent(s), carboxyl, nitro, hydroxyl,

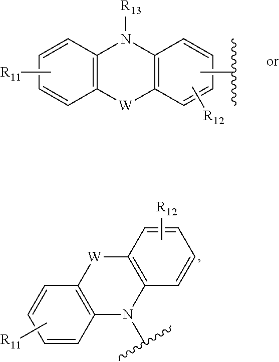

or each of them may be linked to an adjacent substituent via (C3-C30)alkylene or (C3-C30)alkenylene with or without a fused ring to form an aliphatic ring or a monocyclic or polycyclic aromatic ring;

R₁₁ through R₁₃ are the same as defined in R₁ through R₄;

W represents —C(R₅₁R₅₂)ₘ—, —N(R₅₃)—, —S—, —O—, —Si(R₅₄)(R₅₅)—, —P(R₅₆)—, P(═O) (R₅₇)—, —C(═O)—, —B(R₅₈)— or —(R₅₁)C═C(R₅₂)—;

L₁ and L₂ independently represent a chemical bond, (C6-C30)arylene with or without substituent(s), (C3-C30)heteroarylene with or without substituent(s), 5- or 6-membered heterocycloalkylene with or without substituent(s), 5- to 7-membered heterocycloalkylene fused with one or more aromatic ring(s) with or without substituent(s), (C3-C30)cycloalkylene with or without substituent(s), (C3-C30)cycloalkylene fused with one or more aromatic ring(s) with or without substituent(s), adamantylene with or without substituent(s), (C7-C30)bicycloalkylene with or without substituent(s), (C2-C30)alkenylene with or without substituent(s), (C6-C30)ar(C1-C30)alkylene with or without substituent(s) (C1-C30)alkylenethio with or without substituent(s), (C1-C30)alkyleneoxy with or without substituent(s), (C6-C30)aryleneoxy with or without substituent(s), (C6-C30)arylenethio with or without substituent(s), —O—, —S—,

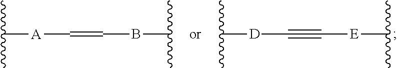

A, B, D and E independently represent a chemical bond, (C6-C30)arylene with or without substituent(s) or (C3-C30)heteroarylene with or without substituent(s);

the heterocycloalkyl or the heteroaryl may contain one or more heteroatom(s) selected from B, N, O, S, P(═O), Si and P; and

m represents an integer 1 or 2.

In the present invention, “alkyl”, “alkoxy” and other substituents containing “alkyl” moiety include both linear and branched species.

In the present invention, “aryl” means an organic radical derived from an aromatic hydrocarbon by the removal of one hydrogen atom, and may include a 4- to 7-membered, particularly 5- or 6-membered, single ring or fused ring, including a plurality of aryls linked by single bond(s). Specific examples include phenyl, naphthyl, biphenyl, anthryl, indenyl, fluorenyl, phenanthryl, triphenylenyl, pyrenyl, perylenyl, chrysenyl, naphthacenyl, fluoranthenyl, etc., but are not limited thereto. The naphthyl includes 1-naphthyl and 2-naphthyl, the anthryl includes 1-anthryl, 2-anthryl and 9-anthryl, and the fluorenyl includes 1-fluorenyl, 2-fluorenyl, 3-fluorenyl, 4-fluorenyl and 9-fluorenyl.

In the present invention, “heteroaryl” means an aryl group containing 1 to 4 heteroatom(s) selected from B, N, O, S, P(═O), Si and P as aromatic ring backbone atom(s), other remaining aromatic ring backbone atoms being carbon. It may be 5- or 6-membered monocyclic heteroaryl or polycyclic heteroaryl resulting from condensation with a benzene ring, and may be partially saturated. Further, the heteroaryl includes more than one heteroaryls linked by single bond(s). The heteroaryl includes a divalent aryl group wherein the heteroatom(s) in the ring may be oxidized or quaternized to form, for example, N-oxide or quaternary salt. Specific examples include monocyclic heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., polycyclic heteroaryl such as benzofuranyl, benzothiophenyl, isobenzofuranyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenanthridinyl, benzodioxolyl, etc., N-oxide thereof (e.g., pyridyl N-oxide, quinolyl N-oxide, etc.), quaternary salt thereof, etc., but are not limited thereto.

In the present invention, the alkyl moiety of “(C1-C30)alkyl, tri(C1-C30)alkylsilyl, di(C1-C30)alkyl(C6-C30)arylsilyl, (C6-C30)ar(C1-C30)alkyl, (C1-C30)alkyloxy, (C1-C30)alkylthio, (C1-C30)alkyloxycarbonyl, (C1-C30)alkylcarbonyl, (C1-C30)alkyloxycarbonyloxy or (C1-C30)alkylcarbonyloxy” may have 1 to 30 carbon atoms, specifically 1 to 20 carbon atoms, more specifically 1 to 10 carbon atoms. The aryl alkyl moiety of “(C6-C30)aryl, di(C1-C30)alkyl(C6-C30)arylsilyl, tri(C6-C30)arylsilyl, (C6-C30)ar(C1-C30)alkyl, (C6-C30)aryloxy, (C6-C30)arylthio, (C6-C30)arylcarbonyl, (C6-C30)aryloxycarbonyl, (C6-C30)arylcarbonyloxy or (C6-C30)aryloxycarbonyloxy” may have 6 to 30 carbon atoms, specifically 6 to 20 carbon atoms, more specifically 6 to 12 carbon atoms. The “(C3-C30)heteroaryl” may have 3 to 30 carbon atoms, specifically 4 to 20 carbon atoms, more specifically 4 to 12 carbon atoms. The “(C3-C30)cycloalkyl” may have 3 to 30 carbon atoms, specifically 3 to 20 carbon atoms, more specifically 3 to 7 carbon atoms. The “(C2-C30)alkenyl or alkynyl” may have 2 to 30 carbon atoms, specifically 2 to 20 carbon atoms, more specifically 2 to 10 carbon atoms.

And, in the present invention, the phrase “with or without substituent(s)” means that the substituents of R₁ through R₄, R₁₁ through R₁₃, R₂₁ through R₂₈, R₅₁ through R₅₈, L₁, L₂, A, B, D and E may be independently substituted with one or more substituent(s) selected from a group consisting of deuterium, halogen, (C1-C30)alkyl with or without halogen substituent(s), (C6-C30)aryl, (C3-C30)heteroaryl with or without (C6-C30)aryl substituent(s), 5- to 7-membered heterocycloalkyl containing one or more heteroatom(s) selected from B, N, O, S, P(═O), Si and P, 5- to 7-membered heterocycloalkyl fused with one or more aromatic ring(s), (C3-C30)cycloalkyl, (C6-C30)cycloalkyl fused with one or more aromatic ring(s), tri(C1-C30)alkylsilyl, di(C1-C30)alkyl(C6-C30)arylsilyl, tri(C6-C30)arylsilyl, adamantyl, (C7-C30)bicycloalkyl, (C2-C30)alkenyl, (C2-C30)alkynyl, cyano, carbazolyl, NR₃₁R₃₂, BR₃₃R₃₄, PR₃₅R₃₆, P(═O)R₃₇R₃₈ [wherein R₃₁ through R₃₈ independently represent (C1-C30)alkyl, (C6-C30)aryl or (C3-C30)heteroaryl], (C6-C30)ar(C1-C30)alkyl, (C1-C30)alkyl(C6-C30)aryl, (C1-C30)alkyloxy, (C1-C30)alkylthio, (C6-C30)aryloxy, (C6-C30)arylthio, (C1-C30)alkoxycarbonyl, (C1-C30)alkylcarbonyl, (C6-C30)arylcarbonyl, (C6-C30)aryloxycarbonyl, (C1-C30)alkoxycarbonyloxy, (C1-C30)alkylcarbonyloxy, (C6-C30)arylcarbonyloxy, (C6-C30)aryloxycarbonyloxy, carboxyl, nitro and hydroxyl, or may be linked to an adjacent substituent to form a ring.

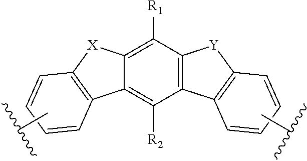

may be selected from the following structures, but is not limited thereto:

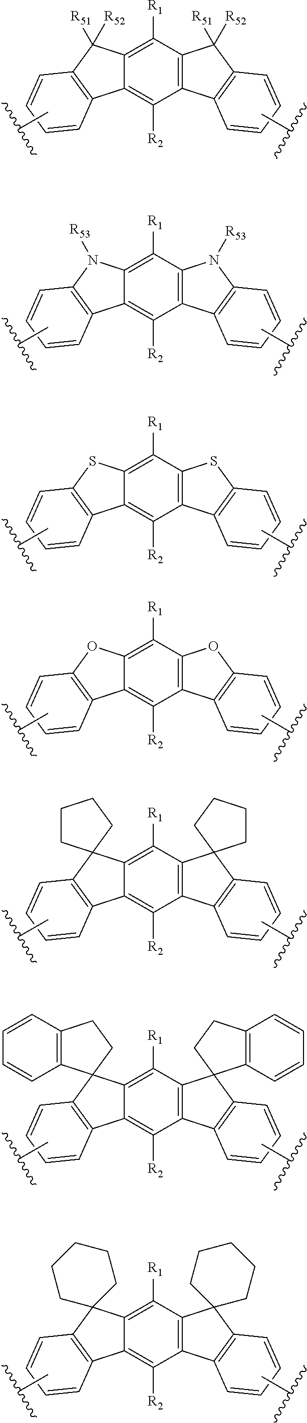

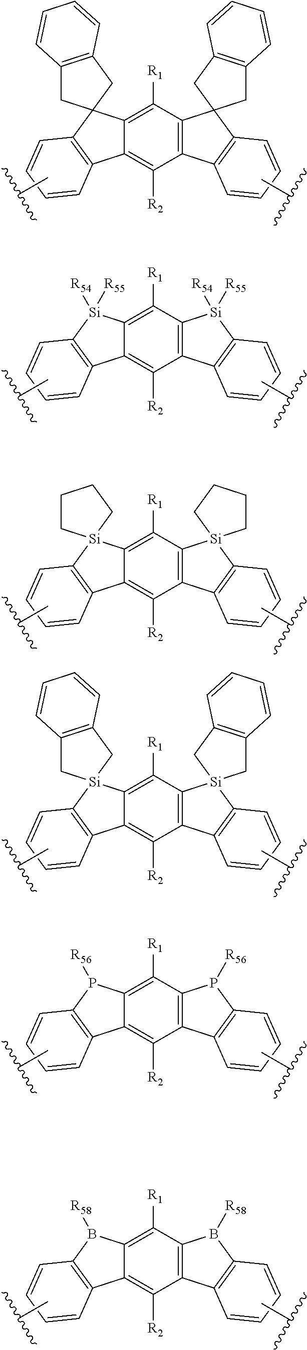

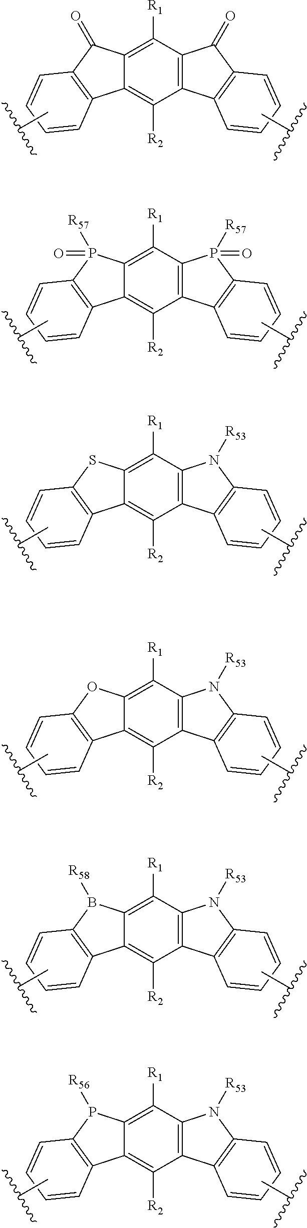

wherein R₁, R₂ and R₅₁ through R₅₈ are independently selected from (C1-C30)alkyl with or without substituent(s), (C6-C30)aryl with or without substituent(s) and (C3-C30)heteroaryl with or without substituent(s).

L₁ and L₂ may be independently selected from a chemical bond, an arylene such as phenylene, naphthylene, anthracenylene, biphenylene, fluorenylene, triphenylenylene, fluoranthenylene, chrysenylene, terphenylene, phenanthrylene, pyrenylene, etc., a heteroarylene such as pyridinylene, pyrazinylene, furylene, thienylene, selenophenylene, quinolinylene, quinoxalinylene, phenanthrolinylene, etc.,

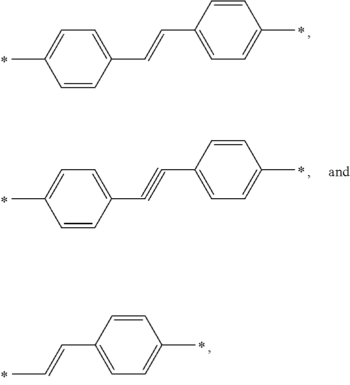

but are not limited thereto. As in Chemical Formula 1, they may be further substituted.

R₃ and R₄ may be independently selected from an aryl such as phenyl, naphthyl, anthryl, biphenyl, fluorenyl, phenanthryl, pyrenyl, perylenyl, etc., a heteroaryl such as pyridinyl, pyrazinyl, furyl, thienyl, selenophenyl, quinolinyl, quinoxalinyl, phenanthrolinyl, carbazolyl, benzopiperidinyl, etc., an aryl fused with cycloalkyl such as tetrahydronaphthyl, etc., a heterocycloalkyl fused with one or more aromatic ring(s) such as benzopiperidino, dibenzomorpholino, dibenzoazepino, etc., NR₂₁R₂₂, BR₂₃R₂₄, PR₂₅R₂₆ and P(═O)R₂₇R₂₈, but are not limited thereto. As in Chemical Formula 1, they may be further substituted.

Specifically,

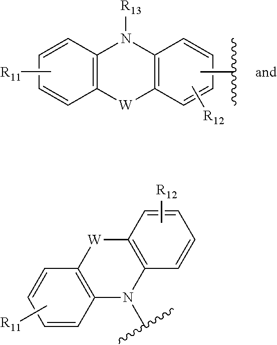

may be exemplified by the following structures:

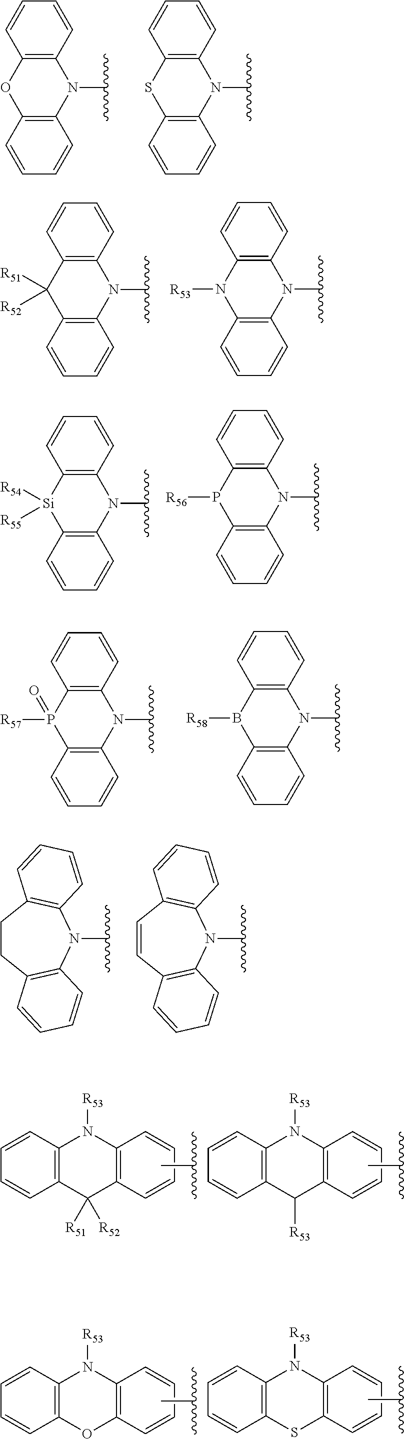

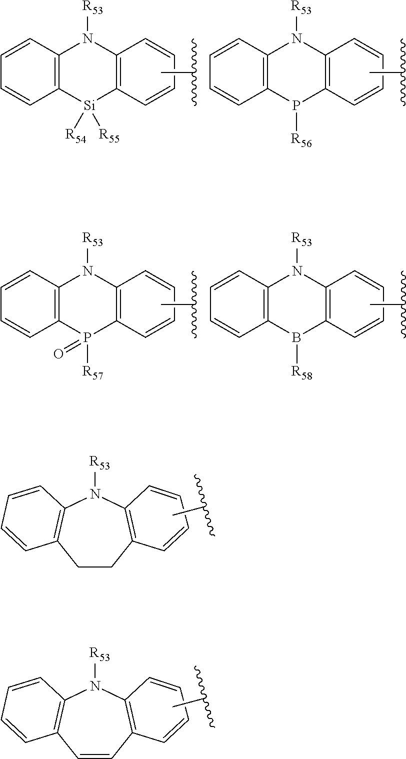

wherein R₅₁ through R₅₈ independently represent (C1-C30)alkyl with or without substituent(s), (C6-C30)aryl with or without substituent(s) or (C3-C30)heteroaryl with or without substituent(s), or each of them may be linked to an adjacent substituent via (C3-C30)alkylene or (C3-C30)alkenylene with or without a fused ring to form an aliphatic ring or a monocyclic or polycyclic aromatic ring.

More specifically, the compound for organic electronic material according to the present invention may be exemplified by the following compounds, but the following compounds do not limit the present invention:

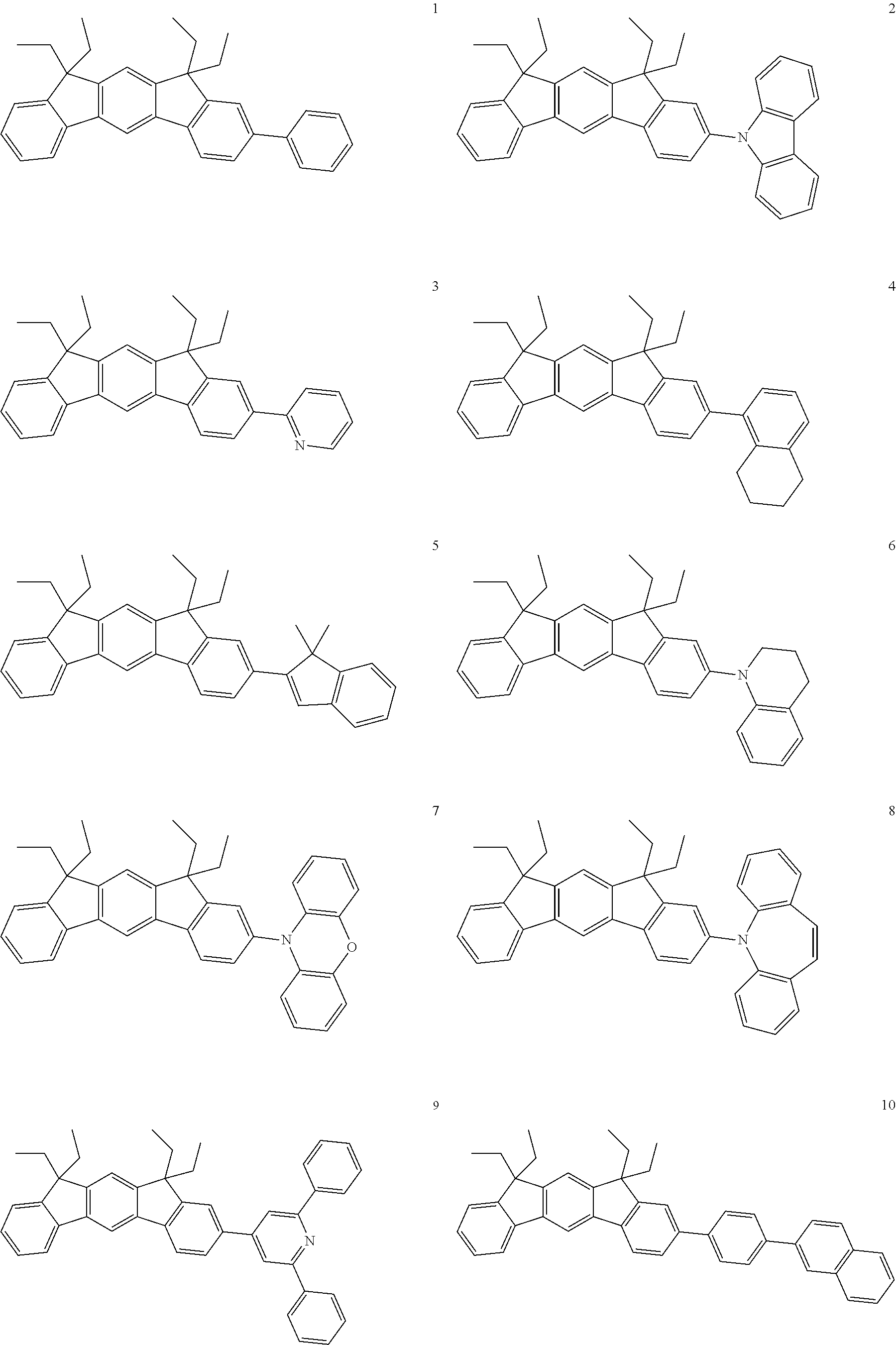

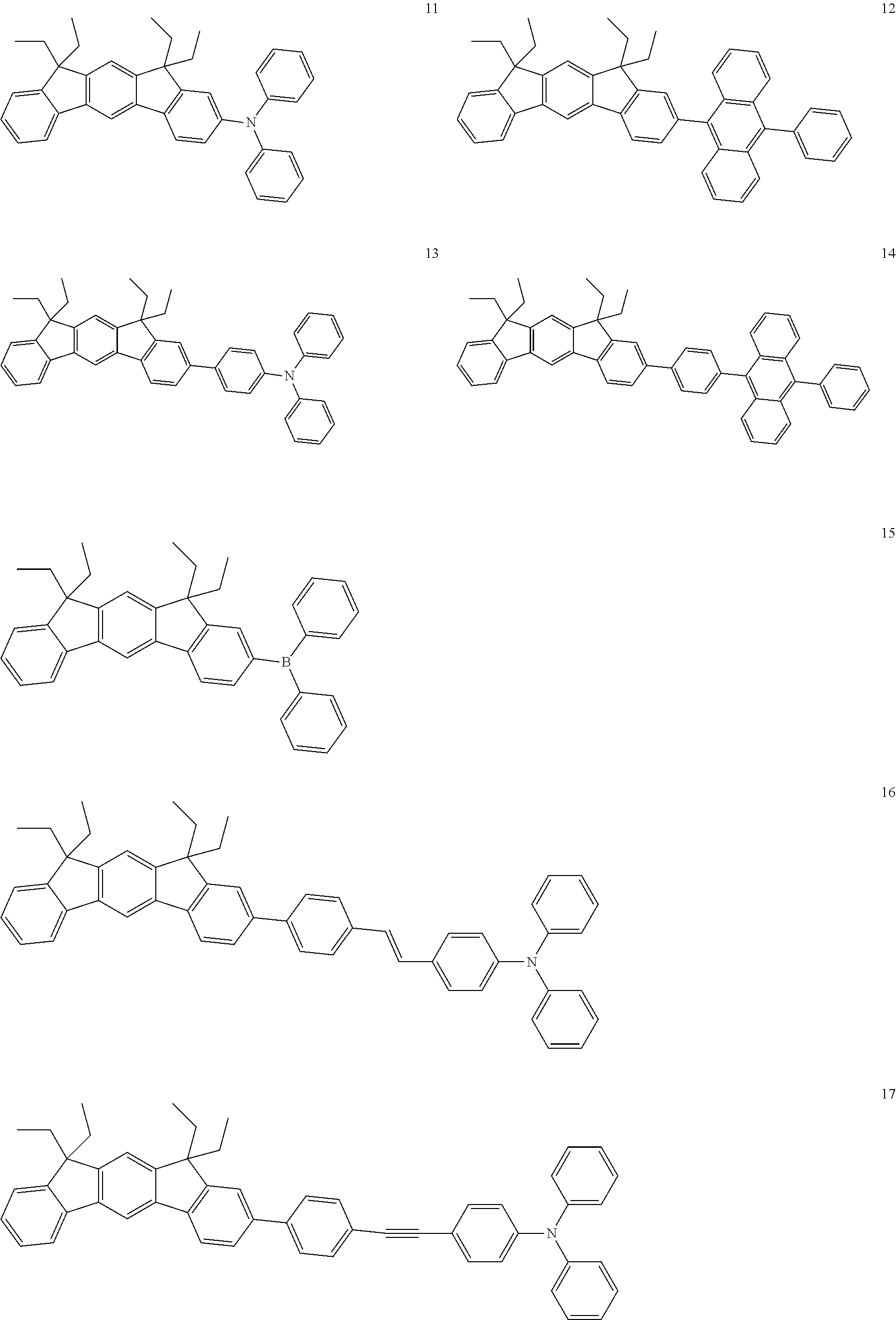

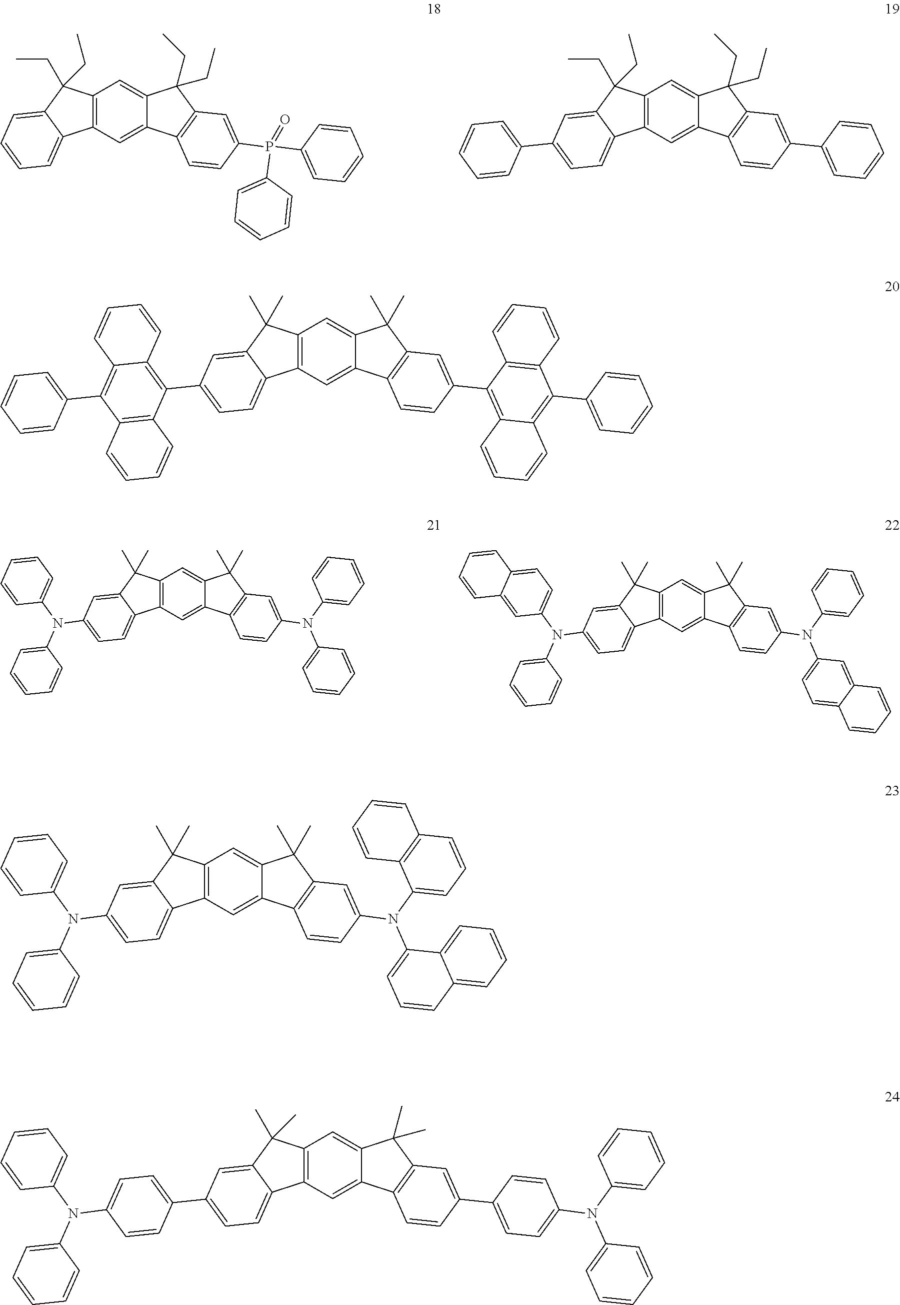

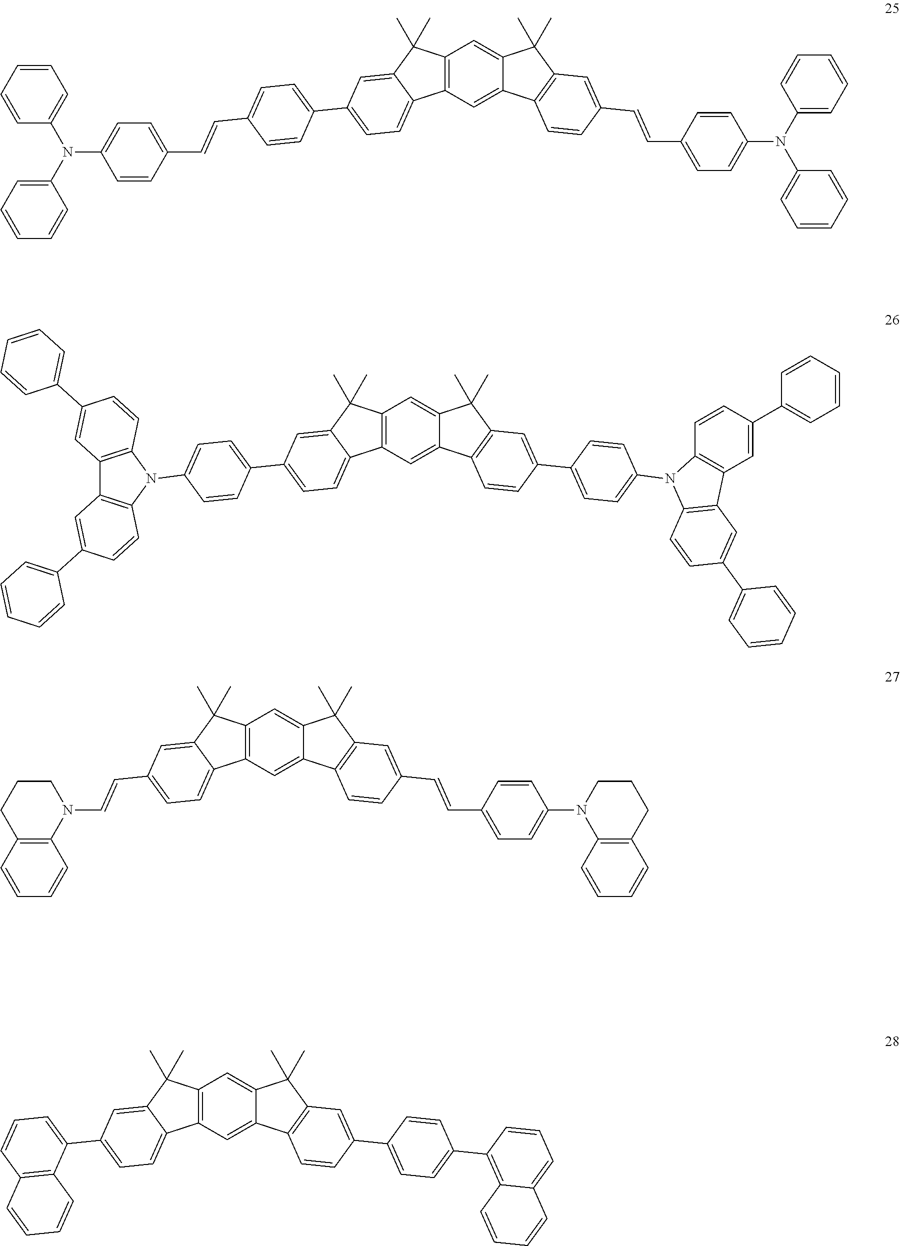

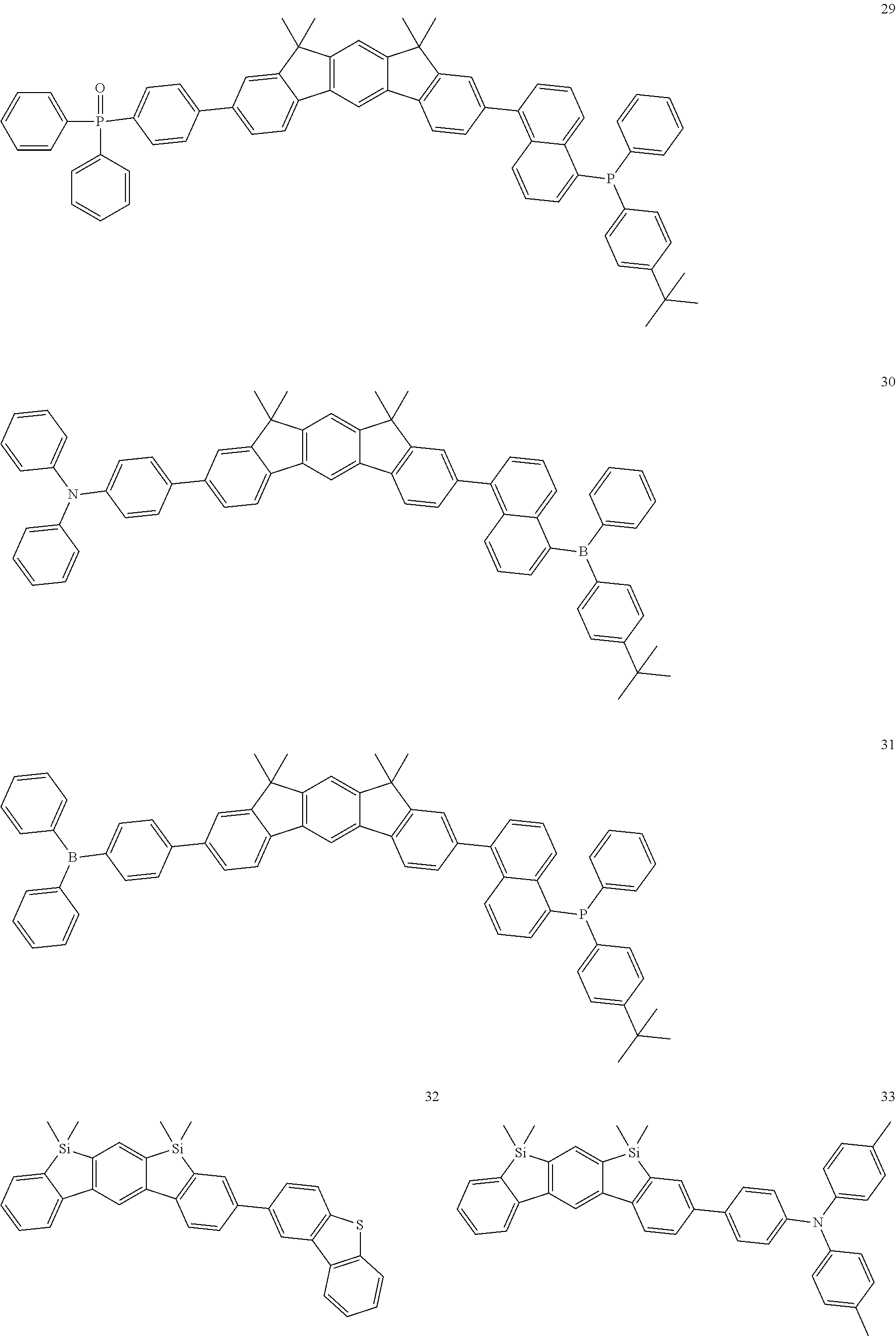

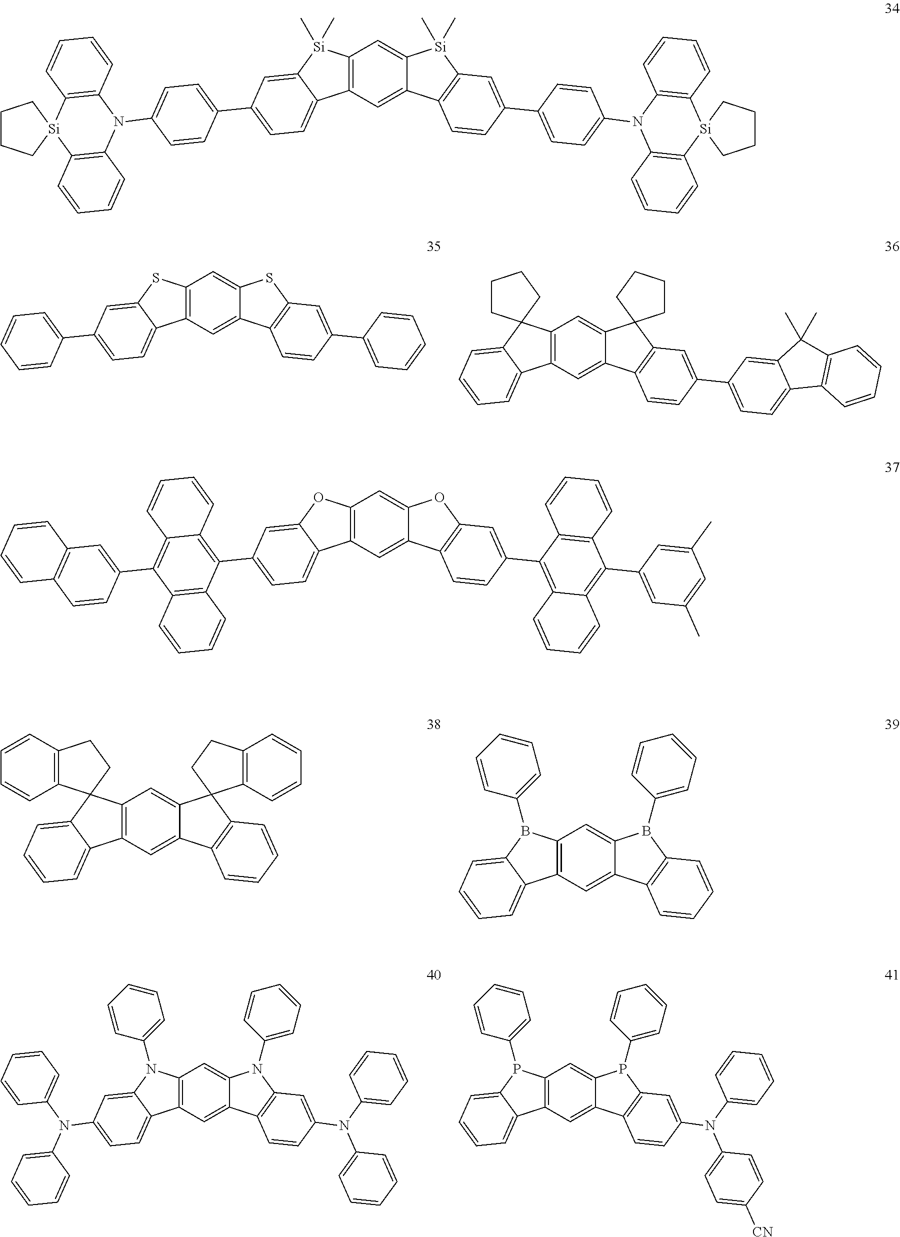

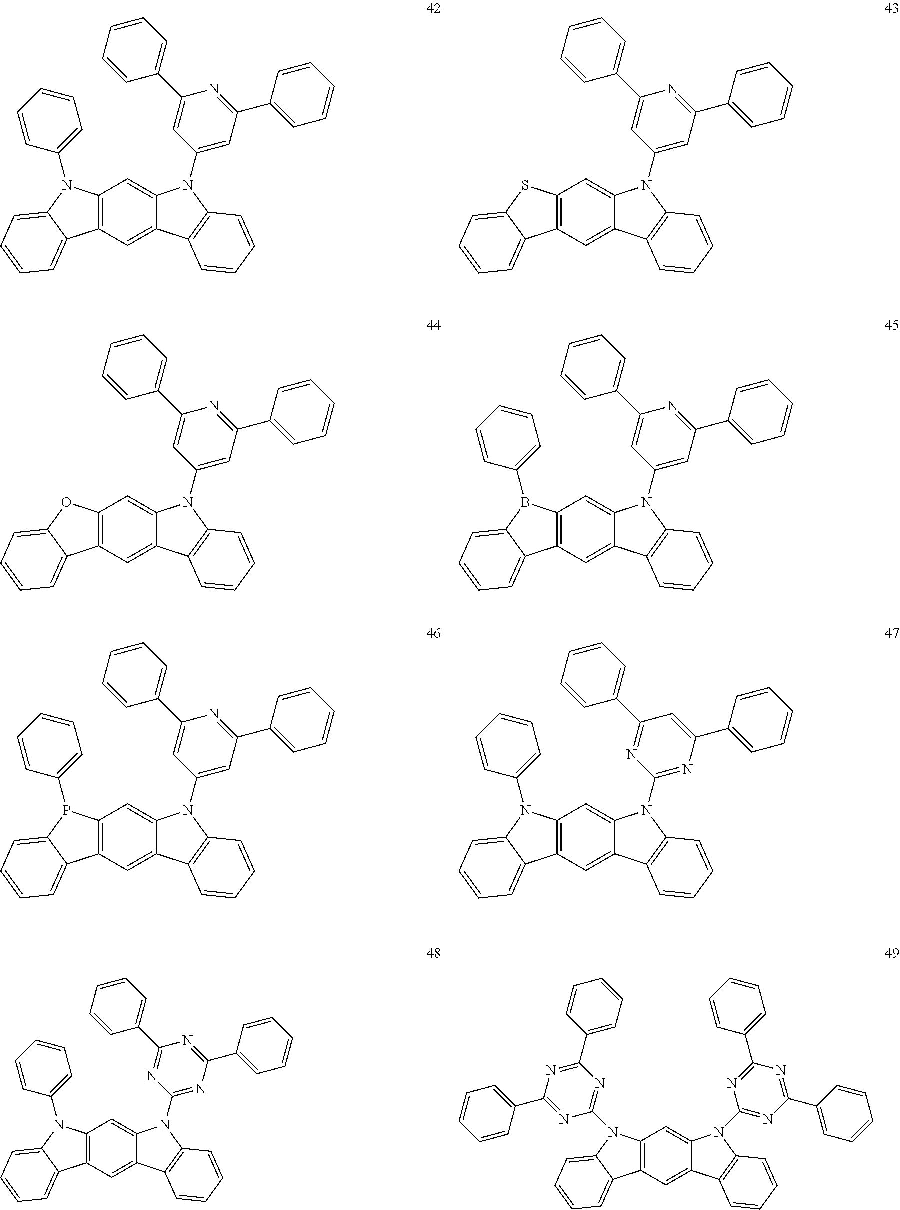

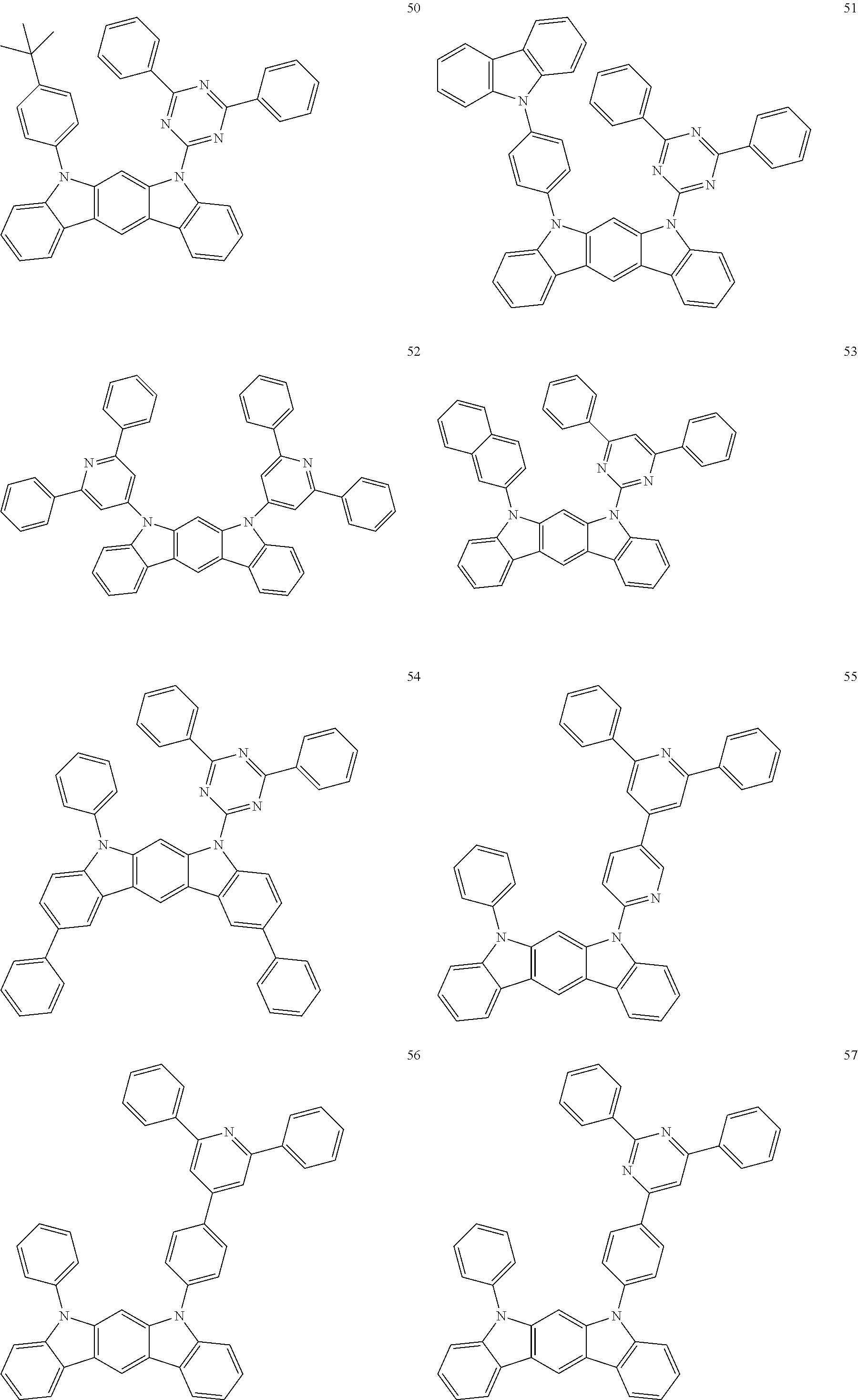

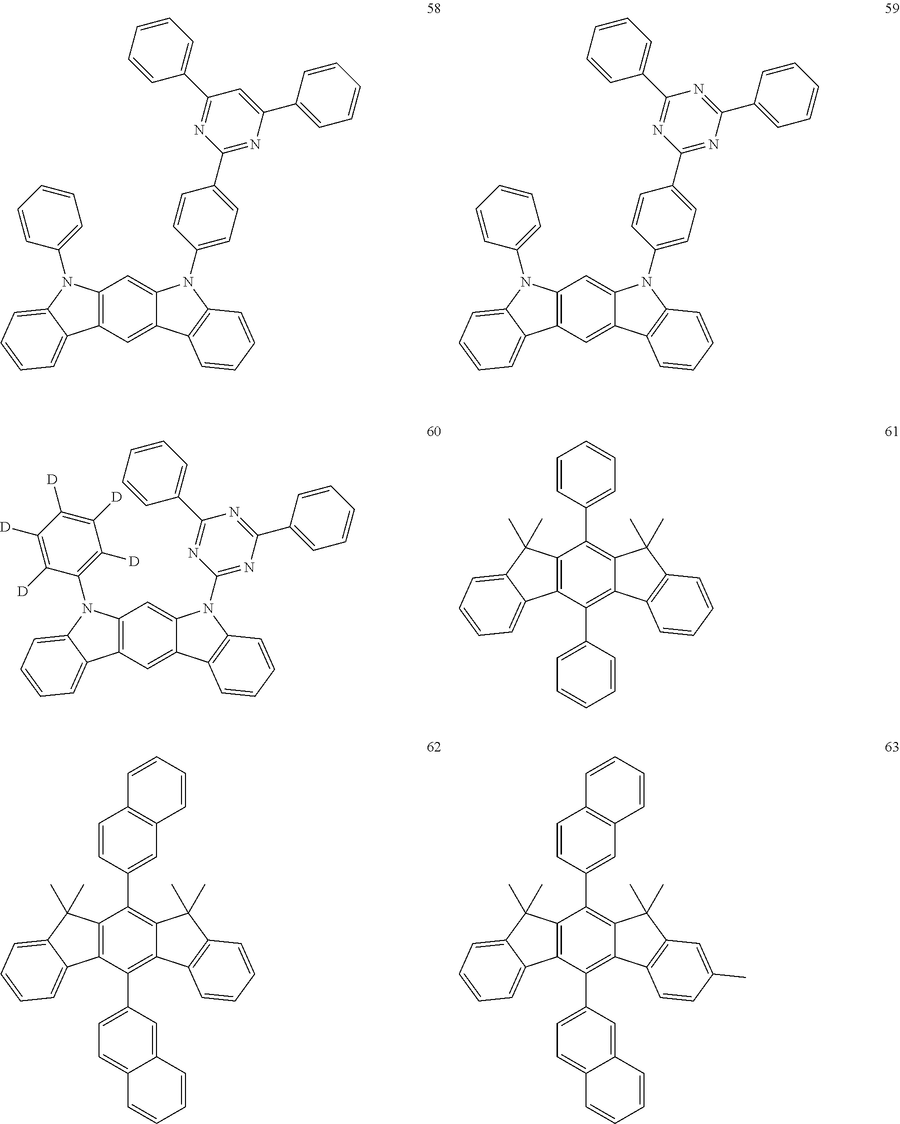

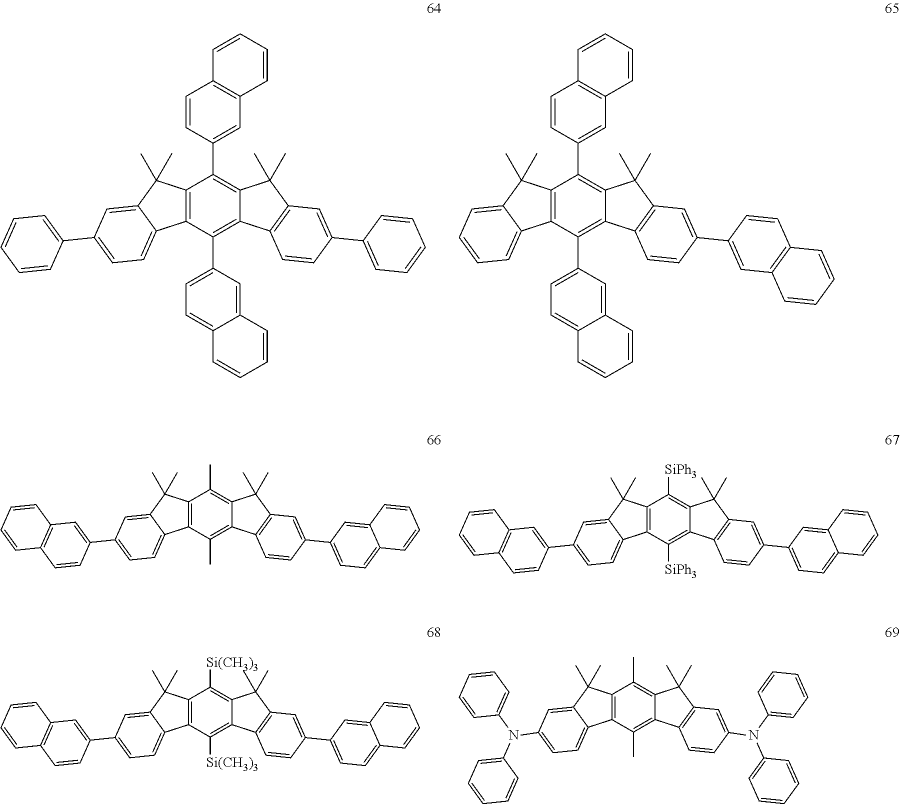

The compound for organic electronic material according to the present invention may be prepared by Scheme 1:

[Scheme 1]

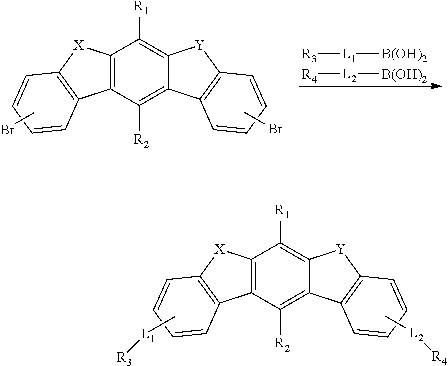

wherein R₁, R₂, R₃, R₄, L₁, L₂, X and Y are the same as defined in Chemical Formula 1.

The present invention provides an organic electronic device including a first electrode; a second electrode; and at least one organic layer(s) interposed between the first electrode and the second electrode. The organic layer includes one or more of the compound(s) for organic electronic material represented by Chemical Formula 1. The compound for organic electronic material may be included in a hole injection layer, a hole transport layer or an electron transport layer, or may be used as a dopant or host material of an electroluminescent layer.

Further, the organic layer may include an electroluminescent layer which further includes one or more dopant(s) or host(s) in addition to one or more of the compound(s) for organic electronic material represented by Chemical Formula 1. The dopant or host used in the organic electronic device of the present invention is not particularly limited.

Preferably, the dopant or host used in the organic electronic device of the present invention is selected from the compounds represented by Chemical Formulas 2 to 6:

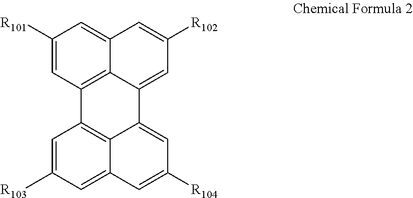

R₁₀₁ through R₁₀₄ independently represent hydrogen, halogen, (C1-C30)alkyl with or without substituent(s), (C6-C30)aryl with or without substituent(s), (C4-C30)heteroaryl with or without substituent(s), 5- or 6-membered heterocycloalkyl with or without substituent(s), 5- to 7-membered heterocycloalkyl fused with one or more aromatic ring(s) with or without substituent(s), (C3-C30)cycloalkyl with or without substituent(s), (C3-C30)cycloalkyl fused with one or more aromatic ring(s) with or without substituent(s), adamantyl with or without substituent(s), (C7-C30)bicycloalkyl with or without substituent(s), cyano, NR₁₁R₁₂, BR₁₃R₁₄, PR₁₅R₁₆, P(═O)R₁₇R₁₈ [wherein R₁₁ through R₁₈ independently represent (C1-C30)alkyl with or without substituent(s), (C6-C30)aryl with or without substituent(s), or (C3-C30)heteroaryl with or without substituent(s).], tri(C1-C30)alkylsilyl with or without substituent(s), di(C1-C30)alkyl(C6-C30)arylsilyl with or without substituent(s), tri(C6-C30)arylsilyl with or without substituent(s), (C6-C30)ar(C1-C30)alkyl with or without substituent(s), (C1-C30)alkyloxy with or without substituent(s), (C1-C30)alkylthio with or without substituent(s), (C6-C30)aryloxy with or without substituent(s), (C6-C30)arylthio with or without substituent(s), (C1-C30)alkoxycarbonyl with or without substituent(s), (C1-C30)alkylcarbonyl with or without substituent(s), (C6-C30)arylcarbonyl with or without substituent(s), (C2-C30)alkenyl with or without substituent(s), (C2-C30)alkynyl with or without substituent(s), (C6-C30)aryloxycarbonyl with or without substituent(s), (C1-C30)alkoxycarbonyloxy with or without substituent(s), (C1-C30)alkylcarbonyloxy with or without substituent(s), (C6-C30)arylcarbonyloxy with or without substituent(s), (C6-C30)aryloxycarbonyloxy with or without substituent(s), carboxyl, nitro or hydroxyl, or each of them may be linked to an adjacent carbon via (C3-C30)alkylene or (C3-C30)alkenylene with or without a fused ring to form a fused ring;

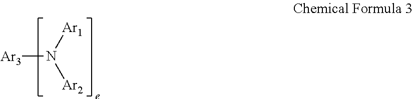

wherein

Ar₁ and Ar₂ independently represent (C1-C30)alkyl with or without substituent(s), (C6-C30)aryl with or without substituent(s), (C4-C30)heteroaryl with or without substituent(s), (C6-C30)arylamino with or without substituent(s), (C1-C30)alkylamino with or without substituent(s), 5- to 7-membered heterocycloalkyl with or without substituent(s), 5- to 7-membered heterocycloalkyl fused with one or more aromatic ring(s) with or without substituent(s), (C3-C30)cycloalkyl with or without substituent(s), or (C3-C30)cycloalkyl fused with one or more aromatic ring(s) with or without substituent(s), or Ar₁ and Ar₂ are linked via (C3-C30)alkylene or (C3-C30)alkenylene with or without a fused ring to form an aliphatic ring or a monocyclic or polycyclic aromatic ring;

in case e is 1, Ar₃ is (C6-C30)aryl with or without substituent(s), (C4-C30)heteroaryl with or without substituent(s) or a substituent selected form the following structures:

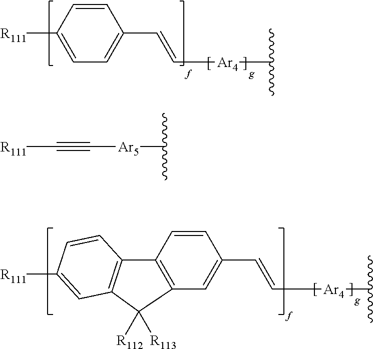

in case e is 2, Ar₃ is (C6-C30)arylene with or without substituent(s), (C4-C30)heteroarylene with or without substituent(s) or a substituent selected form the following structures:

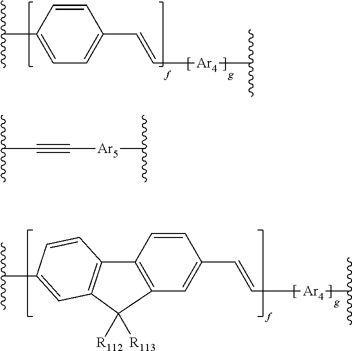

Ar₄ and Ar₅ independently represent (C6-C30)arylene with or without substituent(s) or (C4-C30)heteroarylene with or without substituent(s);

R₁₁₁ through R₁₁₃ independently represent hydrogen, deuterium, (C1-C30)alkyl with or without substituent(s) or (C6-C30)aryl with or without substituent(s);

f is an integer from 1 to 4; and

g is an integer 0 or 1;

M¹L¹⁰¹L¹⁰²L¹⁰³ Chemical Formula 4

wherein

M¹ is selected from a group consisting of Group 7, Group 8, Group 9, Group 10, Group 11, Group 13, Group 14, Group 15 and Group 16 metals;

the ligands L¹⁰¹, L¹⁰² and L¹⁰³ are independently selected from the following structures:

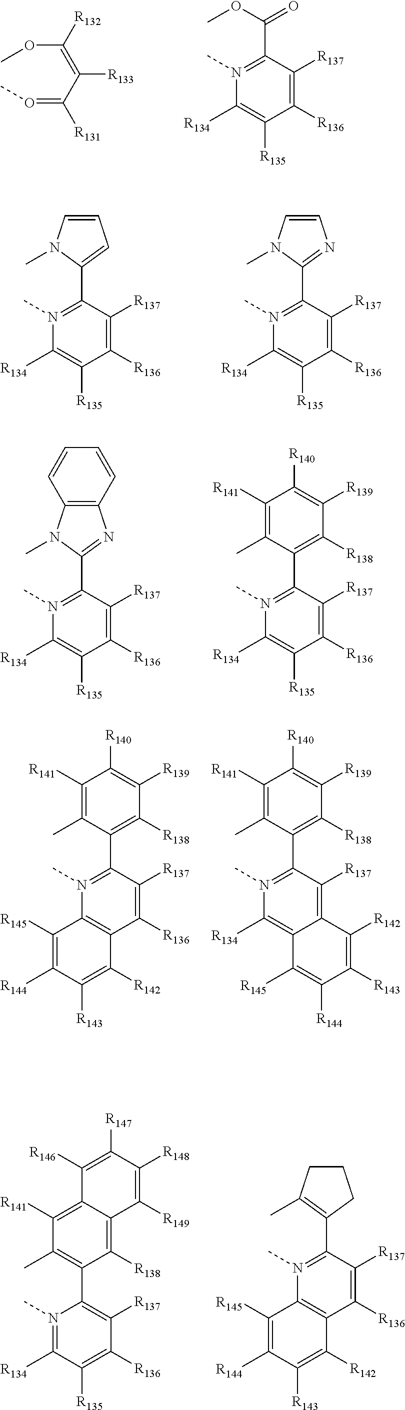

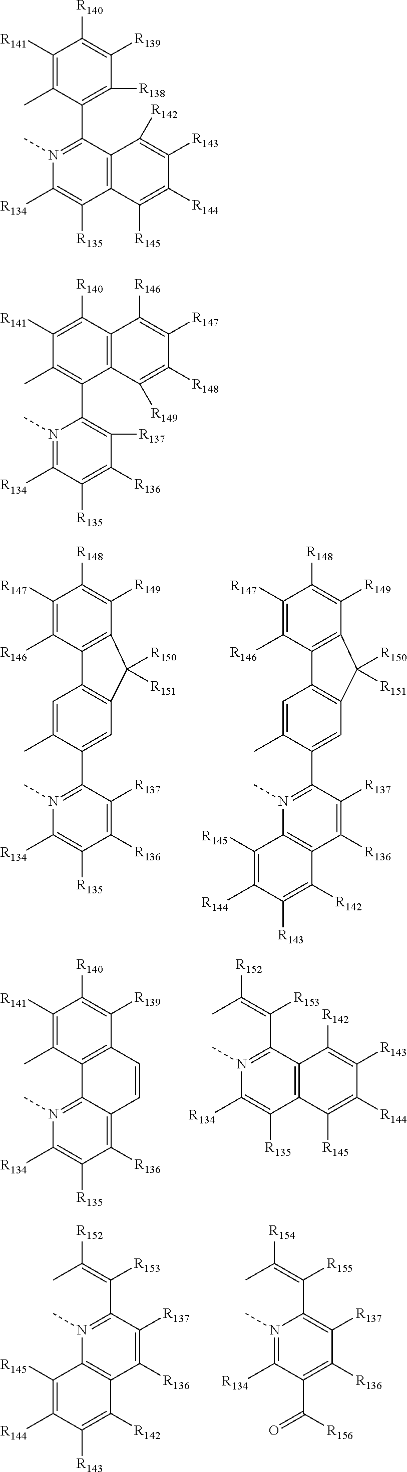

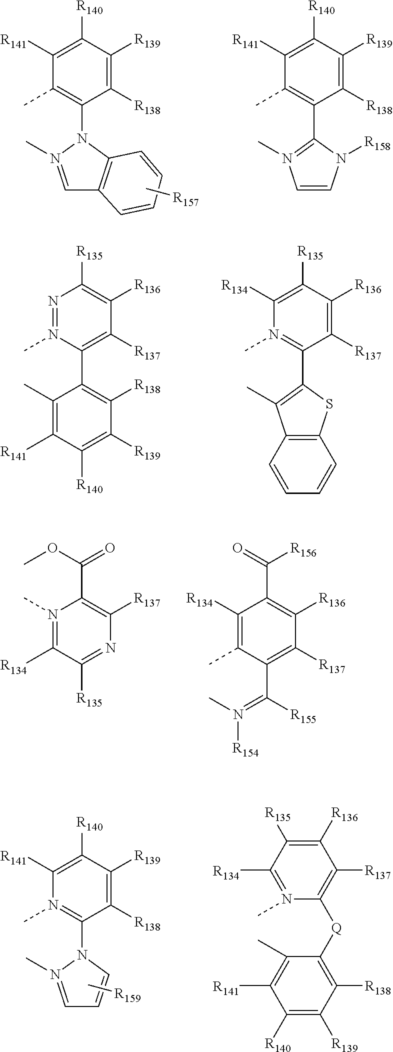

wherein

R₁₃₁ through R₁₃₃ independently represent hydrogen, (C1-C30)alkyl substituted or unsubstituted by halogen(s), (C6-C30)aryl substituted or unsubstituted by (C1-C30)alkyl or halogen;

R₁₃₄ through R₁₄₉ independently represent hydrogen, (C1-C30)alkyl with or without substituent(s), (C1-C30)alkoxy with or without substituent(s), (C3-C30)cycloalkyl with or without substituent(s), (C2-C30)alkenyl with or without substituent(s), (C6-C30)aryl with or without substituent(s), (C1-C30)alkylamino with or without substituent(s), (C6-C30)arylamino with or without substituent(s), SF₅, tri(C1-C30)alkylsilyl with or without substituent(s), di(C1-C30)alkyl(C6-C30)arylsilyl with or without substituent(s), tri(C6-C30)arylsilyl with or without substituent(s), cyano or halogen;

R₁₅₀ through R₁₅₃ independently represent hydrogen, (C1-C30)alkyl substituted or unsubstituted by halogen or (C6-C30)aryl substituted or unsubstituted by (C1-C30)alkyl;

R₁₅₄ and R₁₅₅ independently represent hydrogen, (C1-C30)alkyl with or without substituent(s), (C6-C30)aryl with or without substituent(s) or halogen, or R₁₅₄ and R₁₅₅ are linked via (C3-C12)alkylene or (C3-C12)alkenylene with or without a fused ring to form an aliphatic ring or a monocyclic or polycyclic aromatic ring;

R₁₅₆ represents (C1-C30)alkyl with or without substituent(s), (C6-C30)aryl with or without substituent(s), (C5-C30)heteroaryl with or without substituent(s) or halogen;

R₁₅₇ through R₁₅₉ independently represent hydrogen, (C1-C30)alkyl with or without substituent(s), (C6-C30)aryl with or without substituent(s) or halogen;

Q represents and

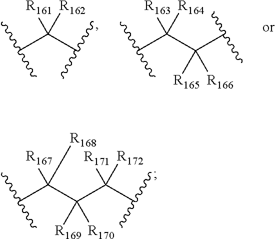

R₁₆₁ through R₁₇₂ independently represent hydrogen, (C1-C30)alkyl substituted or unsubstituted by halogen, (C1-C30)alkoxy, halogen, (C6-C30)aryl with or without substituent(s), cyano or (C5-C30)cycloalkyl with or without substituent(s), or each of them may be linked to an adjacent substituent via alkylene or alkenylene to form a spiro-ring or a fused ring, or each of them may be linked with R₁₃₇ or R₁₃₈ via alkylene or alkenylene to form a fused ring; and

(Ar₁₁)ₕ-L₁₁-(Ar₁₂)ᵢ Chemical Formula 5

(Ar₁₃)ⱼ-L₁₂-(Ar₁₄)ₖ [Chemical Formula 6]

wherein

L₁₁ represents (C6-C30)arylene with or without substituent(s) or (C4-C30)heteroarylene with or without substituent(s);

L₁₂ represents anthracenylene with or without substituent(s);

Ar₁₁ through Ar₁₄ are independently selected from hydrogen, (C1-C30)alkyl with or without substituent(s), (C1-C30)alkoxy with or without substituent(s), halogen, (C4-C30)heteroaryl with or without substituent(s), (C5-C30)cycloalkyl with or without substituent(s) and (C6-C30)aryl with or without substituent(s); and

h, i, j and k are independently an integer from 0 to 4.

In the organic electronic device of the present invention, the organic layer may further include, in addition to the compound for organic electronic material represented by Chemical Formula 1, one or more compound(s) selected from a group consisting of arylamine compounds and styrylarylamine compounds, at the same time. The arylamine compounds or styrylarylamine compounds are exemplified in Korean Patent Application Nos. 10-2008-0123276, 10-2008-0107606 or 10-2008-0118428, but are not limited thereto.

In the organic electronic device of the present invention, the organic layer may further include, in addition to the compound for organic electronic material represented by Chemical Formula 1, one or more metal(s) or complex(es) selected from a group consisting of organic metals of Group 1, Group 2, 4th period and 5th period transition metals, lanthanide metals and d-transition elements. The organic layer may include an electroluminescent layer and a charge generating layer.

Further, the organic layer may include, in addition to the organic electroluminescent compound, one or more organic electroluminescent layer(s) emitting blue, red and green light at the same time, to provide a white light-emitting organic electroluminescent device. The compounds emitting blue, red or green light are exemplified in Korean Patent Application Nos. 10-2008-0123276, 10-2008-0107606 and 10-2008-0118428, but are not limited thereto.

In the organic electroluminescent device of the present invention, a layer (hereinafter referred to as “surface layer”) selected from a chalcogenide layer, a metal halide layer and a metal oxide layer may be placed on the inner surface of one or both electrode(s) among the pair of electrodes. More specifically, a chalcogenide (including oxide) layer of silicon or aluminum may be placed on the anode surface of the electroluminescent medium layer, and a metal halide layer or metal oxide layer may be placed on the cathode surface of the electroluminescent medium layer. A driving stability may be attained therefrom. The chalcogenide may be, for example, SiOₓ (1≦x≦2), AlOₓ (1≦x≦1.5), SiON, SiAlON, etc. The metal halide may be, for example, LiF, MgF₂, CaF₂, a rare earth metal fluoride, etc. The metal oxide may be, for example, Cs₂O, Li₂O, MgO, SrO, BaO, CaO, etc.

Further, in the electroluminescent device according to the present invention, a mixed region of an electron transport compound and a reductive dopant or a mixed region of a hole transport compound and an oxidative dopant may be placed on the inner surface of one or both electrode(s) among the pair of electrodes. In that case, injection and transport of electrons from the mixed region to the electroluminescent medium becomes easier, because the electron transport compound is reduced to an anion. Further, injection and transport of holes from the mixed region to the electroluminescent medium becomes easier, because the hole transport compound is oxidized to a cation.

Preferred examples of the oxidative dopant include various Lewis acids and acceptor compounds. Preferred examples of the reductive dopant include alkali metals, alkali metal compounds, alkaline earth metals, rare earth metals and mixtures thereof.

Further, a white light-emitting organic electroluminescent device having two or more electroluminescent layers may be prepared by using a reductive dopant layer as the charge generating layer.

### Advantageous Effects

Since the compound for organic electronic material according to the present invention exhibits good luminous efficiency and excellent life property, it may be used to manufacture an OLED device having very good operation life.

### BEST MODE

Hereinafter, the compound for organic electronic material, the preparation method thereof and the electroluminescent property of the device according to the present invention will be described for some compounds. However, the following embodiments are only exemplary and do not limit the scope of the present invention.

### Preparation Example 1

### Preparation of Compound 19

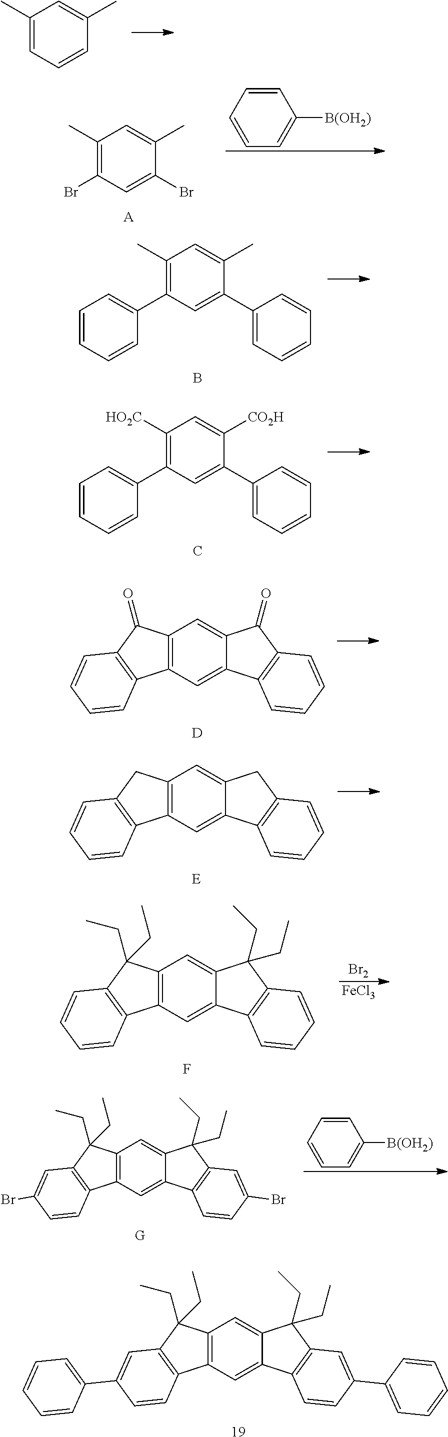

### Preparation of Compound A

1,3-Dimethylbenzene (30.0 g, 282.6 mmol) and FeCl₃ (2.3 g, 14.1 mmol) were dissolved in CCl₄ and Br₂ (32.0 mL, 621.7 mmol) was slowly added thereto at 0° C. After stirring at room temperature for 2 hours, the reaction solution was neutralized with aqueous KOH solution. Extraction with MC followed by drying with MgSO₄, distillation under reduced pressure and column separation yielded Compound A (32.5 g, 123.12 mmol, 43.7%).

### Preparation of Compound B

Compound A (32.5 g, 123.12 mmol), phenylboronic acid (37.5 g, 307.8 mmol), Pd(PPh₃)₄ (5.7 g, 4.9 mmol), toluene (300 mL), ethanol (150 mL) and K₂CO₃ (51.1 g, 369.4 mmol, 2 M aqueous solution) were stirred under reflux. 12 hours later, after cooling to room temperature, the product was extracted with EA, washed with distilled water and dried with MgSO₄. Distillation under reduced pressure followed by column separation yielded Compound B (28.1 g, 108.8 mmol, 88.4%).

### Preparation of Compound C

Compound B (28.1 g, 108.8 mmol) was dissolved in pyridine (500 mL) and KMnO₄ (90.0 g) dissolved in distilled water (60 mL) was added thereto. After stirring for 5 hours under reflux followed by addition of distilled water (500 mL), the mixture was further stirred for 12 hours under reflux. After cooling to room temperature, the resulting solid was filtered. After collecting the filtrate, hydrochloric acid was added until an acidic pH was attained. Filtration of thus produced solid was under reduced pressure followed by drying yielded Compound C (30.7 g, 96.4 mmol, 88.7%).

### Preparation of Compound D

Compound C (30.7 g, 96.4 mmol) was slowly added to sulfuric acid (600 mL). The mixture was stirred at room temperature for 2 hours and ice water was slowly added to the reaction solution. Thus produced purple precipitate was filtered under reduced pressure and washed sequentially with distilled water, K₂CO₃ aqueous solution and distilled water. Compound D (22.4 g, 79.31 mmol, 82.3%) was yielded.

### Preparation of Compound E

KOH (133.5 g, 2380.5 mmol) was added to diethylene glycol (300 mL). After stirring, followed by addition of Compound D (22.4 g, 79.35 mmol) and hydrazine monohydrate (78.9 mL, 1626.6 mmol), the mixture was stirred for 24 hours while heating at 180° C. Upon completion of the reaction, the reaction solution was cooled to room temperature and a solution containing ice in hydrochloric acid was slowly added. Drying of thus produced solid under reduced pressure followed by recrystallization with acetic acid yielded Compound E (17.2 g, 67.62 mmol, 85.2%).

### Preparation of Compound F

Compound E (17.2 g, 67.6 mmol) was dissolved in THF (1.5 L) and cooled to −78° C. Then, n-BuLi (73.0 mL, 182.6 mmol, 2.5 M in hexane) was slowly added. One hour later, bromoethane (15.1 mL, 202.9 mmol) was added. After stirring for an hour, n-BuLi (86.6 mL, 216.4 mmol, 2.5 M in hexane) was slowly added at −78° C. After stirring for an hour, bromoethane (15.1 mL, 202.9 mmol) was added. 5 hours later, distilled water was added and the product was extracted with MC. After drying with MgSO₄, the product was distilled under reduced pressure. Recrystallization with hexane yielded Compound F (14.8 g, 40.4 mmol, 59.7%).

### Preparation of Compound G

Compound F (14.8 g, 40.4 mmol) was dissolved in CHCl₃. After adding FeCl₃ (0.3 g, 2.0 mmol) at 0° C., Br₂ (4.5 mL, 88.8 mmol) was added. After stirring at room temperature for 12 hours, the reaction solution was neutralized with KOH aqueous solution. After extraction with MC, the product was dried with MgSO₄. Distillation under reduced pressure followed by recrystallization with hexane yielded Compound G (15.7 g, 29.9 mmol, 74.9%).

### Preparation of Compound 19

Compound G (15.7 g, 29.9 mmol), phenylboronic acid (9.1 g, 74.9 mmol), Pd(PPh₃)₄ (0.8 g, 1.2 mmol), toluene (200 mL), ethanol (100 mL) and K₂CO₃ (12.4 g, 89.8 mmol, 2 M aqueous solution) were mixed and stirred under reflux. 12 hours later, after cooling to room temperature, methanol was added ant the resulting solid was filtered under reduced pressure. Washing with distilled water and methanol followed by recrystallization with EA and THF yielded Compound 19 (8.5 g, 16.4 mmol, 54.7%).

### Preparation Example 2

### Preparation of Compound 33

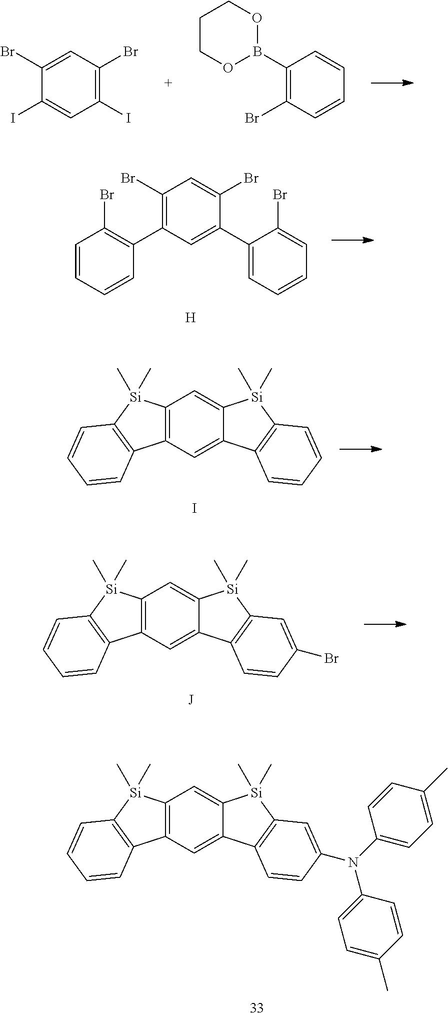

### Preparation of Compound H

1,3-Dibromo-4,6-diiodobenzene (30.0 g, 61.6 mmol), 2-(2-bromophenyl)-1,3,2-dioxaborane (37.0 g, 153.8 mmol), K₃PO₄₇H₂O (31.2 g, 92.3 mmol), Pd(PPh₃)₄ (1.4 g, 1.2 mmol) and DMF were mixed and stirred at 100° C. for 20 hours. After cooling to room temperature, the product was extracted with EA and washed with distilled water. Drying with MgSO₄ followed by distillation under reduced pressure and column separation yielded Compound H (7.3 g, 13.4 mmol, 21.7%).

### Preparation of Compound I

Compound H (7.3 g, 13.4 mmol) was dissolved in diethyl ether (2 L) and n-BuLi (26.7 mL, 66.9 mmol, 2.5 M in hexane) was slowly added at 0° C. After stirring for 4 hours, dichlorodimethylsilane (4.8 mL, 40.1 mmol) was added. After stirring for 12 hours at room temperature, distilled water was added. Extraction with diethyl ether followed by drying with MgSO₄, distillation under reduced pressure and column separation yielded Compound I (1.4 g, 4.1 mmol, 30.6%).

### Preparation of Compound J

Compound I (1.4 g, 4.1 mmol), NBS (0.8 g, 4.5 mmol) and THF (50 mL) were stirred at 0° C. for 8 hours. Upon completion of the reaction, the product was extracted with distilled water and EA. The organic layer was dried with MgSO₄ and the solvent was removed using a rotary evaporator. Separation by column chromatography using hexane and EA as developing solvents yielded Compound J (1.2 g, 2.8 mmol).

### Preparation of Compound 33

Compound J (1.2 g, 2.8 mmol), di-4-methylphenylamine (0.7 g, 4.2 mmol), Pd(OAc)₂ (0.06 g, 0.1 mmol), P(t-Bu)₃ (50% in toluene, 0.09 mL, 0.2 mmol) and Cs₂CO₃ (0.4 g, 8.4 mmol) were dissolved in toluene (50 mL) and stirred at 110° C. for 5 hours under reflux. Upon completion of the reaction, the reaction solution was cooled to room temperature, extracted with EA and distilled water, and dried under reduced pressure. Column separation yielded Compound 33 (0.9 g, 1.7 mmol).

### Preparation Example 3

### Preparation of Compound 40

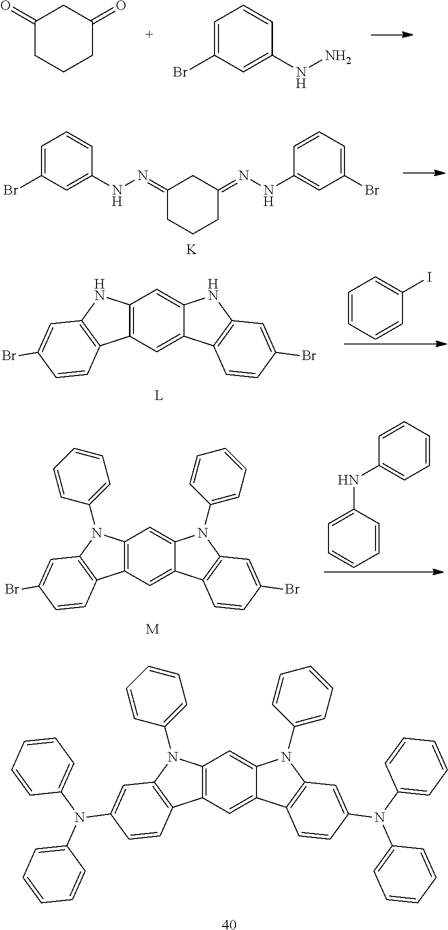

### Preparation of Compound K

3-Bromophenylhydrazine hydrochloride was dissolved in distilled water and 2 M NaOH aqueous solution was added thereto. Thus produced solid was filtered under reduced pressure to obtain 3-bromophenylhydrazine. Cyclohexane-1,3-dione (30.0 g, 267.5 mmol) dissolved in ethanol (1000 mL) was slowly added to 3-bromophenylhydrazine with light blocked. 20 minutes later, the reaction solution was put in ice water. Thus produced solid was filtered under reduced pressure and washed with cold ethanol. Drying under reduced pressure yielded Compound K (46.2 g, 102.6 mmol, 38.4%).

### Preparation of Compound L

Compound K (46.2 g, 102.6 mmol) was slowly added to a mixture solution of acetic acid and sulfuric acid (1:4, 140 mL) at 0° C. After stirring for 5 minutes, the temperature was rapidly raised to 50° C. and then slowly to 110° C. 20 minutes later, after cooling to room temperature, the reaction solution was stirred for 12 hours. After adding ethanol, thus produced solid was filtered under reduced pressure one hour later, and then neutralized. Drying under reduced pressure yielded Compound L (21.7 g, 52.4 mmol, 51.1%).

### Preparation of Compound M

Compound L (21.7 g, 52.4 mmol), iodobenzene (23.4 mL, 209.6 mmol), 18-crown-6 (2.8 g, 10.5 mmol), copper (2.0 g, 31.4 mmol), K₂CO₃ (32.6 g, 235.8 mmol) and 1,2-dichlorobenzene (300 mL) were mixed and stirred at 180° C. for 12 hours. After cooling to room temperature, the reaction solution was distilled under reduced pressure. Extraction with EA followed by washing with distilled water, drying with MgSO₄, distillation under reduced pressure and column separation yielded Compound M (24.3 g, 42.9 mmol, 81.9%).

### Preparation of Compound 40

Compound M (24.3 g, 42.9 mmol), diphenylamine (18.2 g, 107.3 mmol), Pd(OAc)₂ (0.36 g, 1.7 mmol), P(t-Bu)₃ (50% in toluene, 1.5 mL, 3.4 mmol) and Cs₂CO₃ (6.6 g, 128.7 mmol) were dissolved in toluene (500 mL) and stirred at 110° C. for 5 hours under reflux. Upon completion of the reaction, the reaction solution was cooled to room temperature and methanol (1000 mL) was added. Thus produced solid was filtered under reduced pressure and washed with distilled water, methanol and hexane. The solid was mixed with EA (100 mL) and stirred for 2 hours under reflux. After filtration under reduced pressure, the solid was subjected to column separation. The resulting solid was dissolved in THF and methanol was added. Filtration of the resulting solid under reduced pressure yielded Compound 40 (15.3 g, 20.6 mmol).

### Preparation Example 4

### Preparation of Compound 46

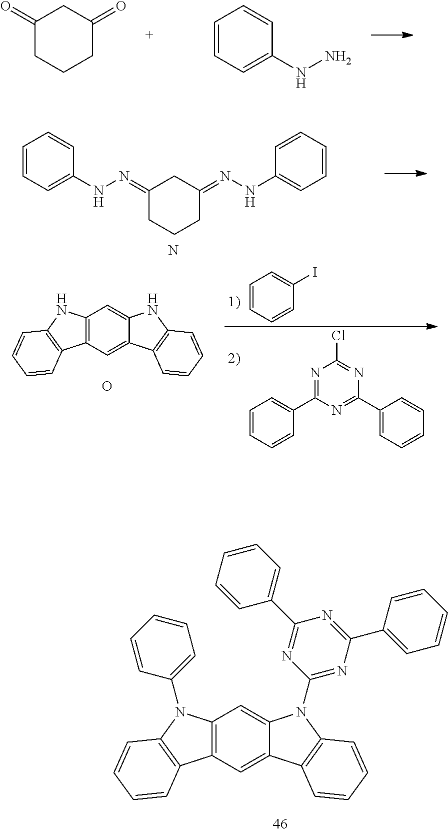

### Preparation of Compound N

Phenylhydrazine hydrochloride was dissolved in distilled water and 2 M NaOH aqueous solution was added thereto. Thus produced solid was filtered under reduced pressure to obtain phenylhydrazine. Cyclohexane-1,3-dione (30.0 g, 267.5 mmol) dissolved in ethanol (1000 mL) was slowly added to phenylhydrazine with light blocked. 20 minutes later, the reaction solution was put in ice water. Thus produced solid was filtered under reduced pressure and washed with cold ethanol. Drying under reduced pressure yielded Compound N (46.2 g, 102.6 mmol, 38.4%).

### Preparation of Compound O

Compound N (46.2 g, 102.6 mmol) was slowly added to a mixture solution of acetic acid and sulfuric acid (1:4, 140 mL) at 0° C. After stirring for 5 minutes, the temperature was rapidly raised to 50° C. and then slowly to 110° C. 20 minutes later, after cooling to room temperature, the reaction solution was stirred for 12 hours. After adding ethanol, thus produced solid was filtered under reduced pressure one hour later, and then neutralized. Drying under reduced pressure yielded Compound O (21.7 g, 52.4 mmol, 51.1%).

### Preparation of Compound 46

Compound O (10.0 g, 39.0 mmol), iodobenzene (5.2 mL, 46.8 mmol), 18-crown-6 (2.1 g, 7.8 mmol), copper (1.5 g, 23.4 mmol), K₂CO₃ (24.3 g, 175.5 mmol) and 1,2-dichlorobenzene (150 mL) were mixed and stirred at 180° C. for 5 hours. Then, 2-chloro-4,6-diphenyl-1,3,5-triazine (12.5 g, 46.8 mmol), 18-crown-6 (2.1 g, 7.8 mmol) and copper (1.5 g, 23.4 mmol) were added. After stirring at 180° C. for 12 hours and cooling to room temperature, the reaction solution was extracted with EA and washed with distilled water. Drying with MgSO₄ followed by distillation under reduced pressure and column separation yielded Compound 46 (3.8 g, 6.7 mmol, 17.30).

Organic electroluminescent compounds, Compounds 1 to 69, were prepared in the same manner as Preparation Examples 1 to 4. ¹H NMR and MS/FAB data of thus prepared organic electroluminescent compounds are given in Table 1.

TABLE 1

| | | MS/FAB | |
|---|---|---|---|
| Compound | ¹H NMR(CDCl₃, 200 MHz) | found | calculated |
| 1 | δ = 1.72(12H, s), 7.24(1H, m), 7.41~7.44(2H, m), 7.48(1H, s), | 386.53 | 386.20 |
| | 7.51~7.52(4H, m), 7.61~7.63(2H, m), 7.77(1H, m), 7.93(1H, | | |
| | m), 7.98(1H, s), 8.09(1H, m) | | |
| 6 | δ = 1.72(12H, s), 1.96(2H, m), 2.76(2H, m), 3.06(2H, m), | 441.61 | 441.25 |
| | 6.55(2H, m), 6.72(2H, m), 7.05~7.07(2H, m), 7.24(1H, m), | | |
| | 7.44(1H, m), 7.48(1H, s), 7.61~7.65(2H, m), 7.98(1H, s), | | |
| | 8.09(1H, m) | | |
| 11 | δ = 1.72(12H, s), 6.58~6.63(5H, m), 6.75~6.81(3H, m), | 477.64 | 477.25 |
| | 7.2~7.24(5H, m), 7.44(1H, m), 7.48(1H, s), 7.61~7.62(2H, m), | | |
| | 7.98(1H, s), 8.09(1H, m) | | |
| 12 | δ = 1.72(12H, s), 7.24(1H, m), 7.39~7.44(6H, m), 7.48(1H, s), | 562.74 | 562.27 |
| | 7.51~7.52(4H, m), 7.61~7.63(2H, m), 7.77(1H, m), | | |
| | 7.91~7.93(5H, m), 7.98(1H, s), 8.09(1H, m) | | |
| 13 | δ = 1.72(12H, s), 6.63(4H, m), 6.69(2H, m), 6.81(2H, m), | 553.73 | 553.28 |
| | 7.2~7.24(5H, m), 7.44(1H, m), 7.48(1H, s), 7.54(2H, m), | | |
| | 7.61~7.63(2H, m), 7.77(1H, m), 7.93(1H, m), 7.98(1H, s), | | |
| | 8.09(1H, m) | | |
| 16 | δ = 1.72(12H, s), 6.63(6H, m), 6.81(2H, m), 6.95(2H, m), | 655.87 | 655.32 |
| | 7.2~7.24(5H, m), 7.44(1H, m), 7.48(1H, s), 7.56~7.64(6H, m), | | |
| | 7.77(3H, m), 7.93(1H, m), 7.98(1H, s), 8.09(1H, m) | | |
| 17 | δ = 1.72(12H, s), 6.59~6.63(6H, m), 6.81(2H, m), | 653.85 | 653.31 |
| | 7.2~7.24(5H, m), 7.34(2H, m), 7.44(1H, m), 7.48(1H, s), | | |
| | 7.61~7.65(6H, m), 7.77(1H, m), 7.93(1H, m), 7.98(1H, s), | | |
| | 8.09(1H, m) | | |
| 20 | δ = 1.72(12H, s), 7.39~7.41(10H, m), 7.48(1H, s), | 815.05 | 814.36 |
| | 7.51~7.52(8H, m), 7.66(2H, m), 7.8(2H, m), 7.91(8H, m), | | |
| | 7.98(1H, s), 8.04(2H, m) | | |
| 21 | δ = 1.72(12H, s), 6.61~6.63(10H, m), 6.78~6.81(6H, m), | 644.84 | 644.32 |
| | 7.2(8H, m), 7.48(1H, s), 7.73(2H, m), 7.98(1H, s), (H,) | | |
| 24 | δ = 1.72(12H, s), 6.63(8H, m), 6.69(4H, m), 6.81(4H, m), | 797.04 | 796.38 |
| | 7.2(8H, m), 7.48(1H, s), 7.54(4H, m), 7.66(2H, m), 7.8(2H, m), | | |
| | 7.98(1H, s), 8.04(2H, m) | | |
| 25 | δ = 1.72(12H, s), 6.63(12H, m), 6.81(4H, m), 6.95(4H, m), | 1001.30 | 1000.48 |
| | 7.2(8H, m), 7.48(1H, s), 7.56(4H, m), 7.64~7.66(6H, m), | | |
| | 7.77~7.8(6H, m), 7.98(1H, s), 8.04(2H, m) | | |
| 28 | δ = 1.72(12H, s), 7.47(2H, m), 7.48(1H, s), 7.54(4H, m), | 616.79 | 616.29 |
| | 7.63(1H, m), 7.69(1H, m), 7.77(1H, m), 7.83(1H, m), 7.93(1H, | | |
| | m), 7.98(1H, s), 7.99(2H, m), 8.15~8.2(3H, m), 8.3(4H, m), | | |
| | 8.75(2H, m) | | |
| 42 | δ = 7.05(2H, m), 7.25~7.33(3H, m), 7.4(1H, s), | 561.67 | 561.22 |
| | 7.45~7.54(10H, m), 7.55(1H, s), 7.58~7.63(3H, m), 7.94(1H, | | |
| | m), 8.12(1H, m), 8.3(4H, m), 8.55(1H, m) | | |
| 48 | δ = 7.25~7.33(3H, m), 7.4(1H, s), 7.41~7.51(10H, m), | 563.65 | 563.21 |
| | 7.55(1H, s), 7.58~7.63(3H, m), 7.94(1H, m), 8.12(1H, m), | | |
| | 8.28(4H, m), 8.55(1H, m) | | |
| 49 | δ = 7.25~7.33(3H, m), 7.4(1H, s), 7.41(4H, m), 7.5~7.51(9H, | 718.81 | 718.26 |
| | m), 7.55(1H, s), 7.63(1H, m), 7.94(1H, m), 8.12(1H, m), | | |
| | 8.28(8H, m), 8.55(1H, m) | | |
| 54 | δ = 7.4(1H, s), 7.41~7.52(19H, m), 7.55(1H, s), 7.58(2H, m), | 715.84 | 715.27 |
| | 7.69(1H, m), 7.77(2H, m), 7.87(1H, m), 8(1H, m), 8.18(1H, m), | | |
| | 8.28(4H, m) | | |
| 59 | δ = 7.25~7.33(3H, m), 7.4(1H, s), 7.41~7.51(10H, m), | 639.75 | 639.24 |
| | 7.55(1H, s), 7.58~7.68(5H, m), 7.79(2H, m), 7.94(1H, m), | | |
| | 8.12(1H, m), 8.28(4H, m), 8.55(1H, m) | | |
| 62 | δ = 1.72(12H, s), 7.26(2H, m), 7.41(2H, m), 7.58~7.59(8H, m), | 562.74 | 562.27 |
| | 7.73(2H, m), 7.92(2H, m), 7.98~8(6H, m) | | |
| 65 | δ = 1.72(12H, s), 7.24(1H, m), 7.44(1H, m), 7.58~7.63(11H, | 688.90 | 688.31 |
| | m), 7.73~7.77(4H, m), 7.92~7.93(4H, m), 8(6H, m), 8.09(1H, | | |
| | m) | | |
| 69 | δ = 1.72(12H, s), 2.18(3H, s), 2.34(3H, s), 6.61~6.63(10H, m), | 672.90 | 672.35 |
| | 6.78~6.81(6H, m), 7.2(8H, m), 7.73(2H, m) | | |

### Example 1

### Manufacture of OLED Device Using the Compound for Organic Electronic Material According to the Present Invention

An OLED device was manufactured using the compound for electronic material of the present invention.

First, a transparent electrode ITO film (15Ω/□) prepared from a glass substrate for an OLED (Samsung Corning) was subjected to ultrasonic washing sequentially using trichloroethylene, acetone, ethanol and distilled water, and stored in isopropanol for later use.

Then, the ITO substrate was mounted on a substrate holder of a vacuum deposition apparatus. After adding 4,4′,4″-tris(N,N-(2-naphthyl)-phenylamino)triphenylamine (2-TNATA) in a cell of the vacuum deposition apparatus, the pressure inside the chamber was reduced to 10⁻⁶ torr. Then, 2-TNATA was evaporated by applying electrical current to the cell to form a hole injection layer having a thickness of 60 nm on the ITO substrate. Subsequently, after adding N,N′-bis(α-naphthyl)-N,N′-diphenyl-4,4′-diamine (NPB) in another cell of the vacuum deposition apparatus, NPB was evaporated by applying electrical current to the cell to form a hole transport layer having a thickness of 20 nm on the hole injection layer.

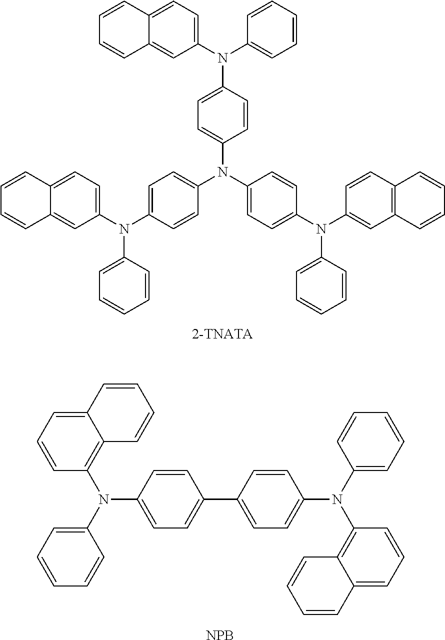

An electroluminescent layer was formed on the hole transport layer as follows. The compound according to the present invention (e.g. Compound 1) was added in a cell of a vacuum deposition apparatus as an electroluminescent material, and DSA-Ph was added in another cell. The two cells were heated together such that an electroluminescent layer having a thickness of 30 nm was formed on the hole transport layer at 2 to 5 wt % based on DSA-Ph.

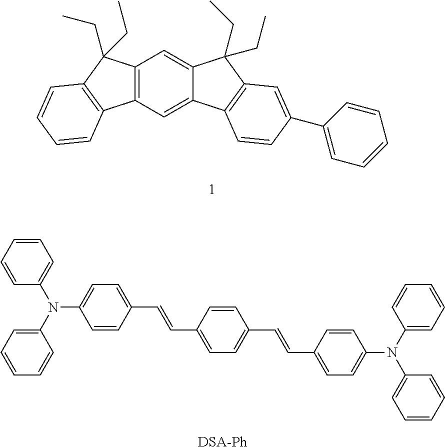

Thereafter, tris(8-hydroxyquinoline)-aluminum(III) (Alq) was deposited with a thickness of 20 nm on the electroluminescent layer as an electron transport layer, and lithium quinolate (Liq) was deposited with a thickness of 1 to 2 nm as an electron injection layer. Then, an Al cathode having a thickness of 150 nm was formed using another vacuum deposition apparatus to manufacture an OLED.

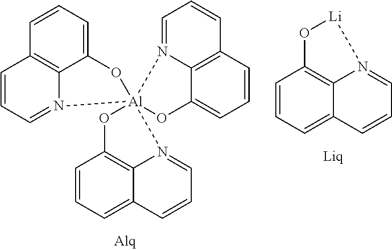

Each OLED electroluminescent used in the OLED device had been purified by vacuum sublimation at 10⁻⁶ torr.

### Example 2

### Manufacture of OLED Device Using the Compound for Organic Electronic Material According to the Present Invention

A hole injection layer and a hole transport layer were formed in the same manner as Example 1, and then an electroluminescent layer was formed thereon as follow. Dinaphthylanthracene (DNA) was added in a cell of a vacuum deposition apparatus as a host, and Compound 24 according to the present invention was added in another cell as a dopant. The two cells were evaporated at different rate such that an electroluminescent layer having a thickness of 30 nm was formed on the hole transport layer at 2 to 5 wt % based on the host.

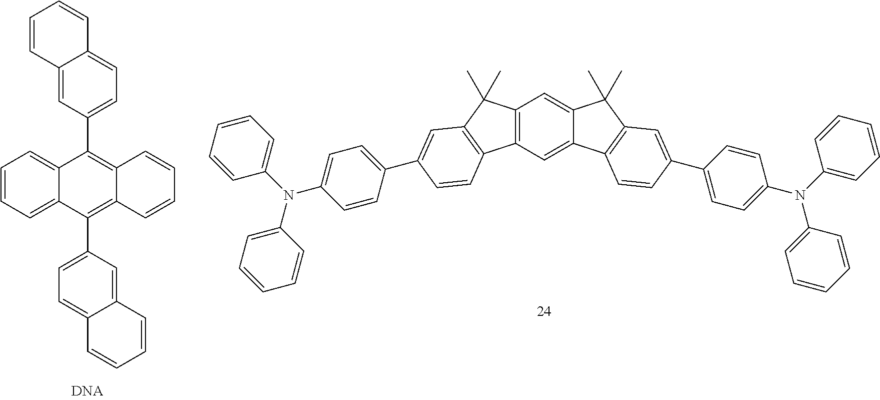

Subsequently, an electron transport layer and an electron injection layer were formed in the same manner as Example 1, and an Al cathode having a thickness of 150 nm was formed using another vacuum deposition apparatus to manufacture an OLED.

### Comparative Example 1

### Electroluminescent Property of OLED Device Using Existing Electroluminescent Material

A hole injection layer and a hole transport layer were formed in the same manner as Example 1. Then, after adding dinaphthylanthracene (DNA) in a cell of the vacuum deposition apparatus as an electroluminescent host material and adding DSA-Ph in another cell as in Example 1, the two materials were evaporated at different rate of 100:3 such that an electroluminescent layer having a thickness of 30 nm was formed on the hole transport layer.

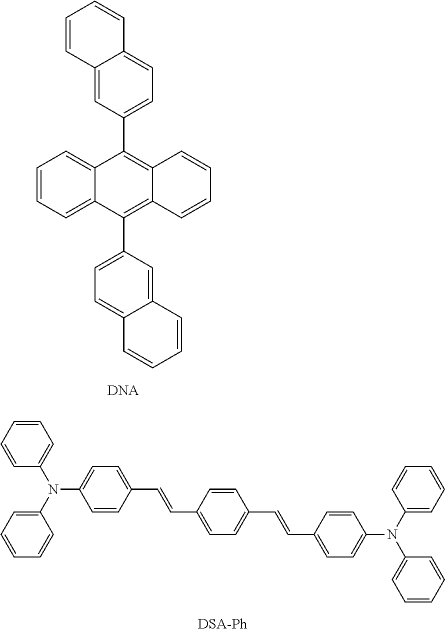

Subsequently, after forming an electron transport layer and an electron injection layer in the same manner as Example 1, an Al cathode having a thickness of 150 nm was formed using another vacuum deposition apparatus to manufacture an OLED.

Luminous efficiency of the OLED devices manufactured in Examples 1 and 2 and Comparative Example 1 was measured at 1,000 cd/m². The result is given in Table 2.

TABLE 2

| | | | | Luminous | |
|---|---|---|---|---|---|
| | | | Doping | efficiency | |
| | | | concentration | (cd/A) @ | Emitted |
| No. | Host | Dopant | (wt %) | 1,000 cd/m² | color |
| Ex. 1 | 1 | DSA-Ph | 3 | 13.0 | Light blue |
| | 3 | DSA-Ph | 3 | 12.8 | Light blue |
| | 12 | DSA-Ph | 3 | 13.2 | Light blue |
| | 38 | DSA-Ph | 3 | 12.9 | Light blue |
| Ex. 2 | DNA | 24 | 3 | 12.7 | Blue |
| | DNA | 25 | 3 | 12.5 | Blue |
| | DNA | 26 | 3 | 12.6 | Blue |
| | DNA | 30 | 3 | 12.7 | Blue |
| Comp. Ex. 1 | DNA | DSA-Ph | 3 | 12.4 | Light blue |

As seen from Table 2, when applied to a blue light-emitting electroluminescent device as host, the organic electroluminescent compounds according to the present invention exhibit comparable or better luminous efficiency as compared to Comparative Example 1. Further, when they were used as dopant, they exhibit comparable or better luminous efficiency as well as significantly improved color purity, as compared to Comparative Example 1.

### Example 3

### Manufacture of OLED Device Using the Compound for Organic Electronic Material According to the Present Invention

A hole injection layer was formed in the same manner as Comparative Example 1. Subsequently, after adding Compound 22 in another cell of the vacuum deposition apparatus, it was evaporated by applying electrical current to the cell to form a hole transport layer having a thickness of 20 nm on the hole injection layer.

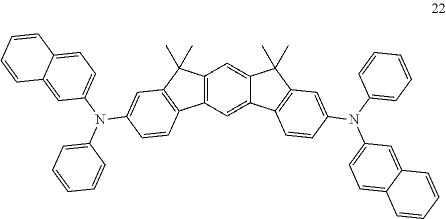

An OLED device was manufactured with other conditions the same as those of Comparative Example 1.

Luminous efficiency of the OLED devices manufactured in Example 3 and Comparative Example 1 was measured at 1,000 cd/m². The result is given in Table 3.

TABLE 3

| | | | Luminous |
|---|---|---|---|
| | Hole | | efficiency |
| | transport | Driving voltage | (cd/A) @ |
| No. | material | (V) @ 1,000 cd/m² | 1,000 cd/m² |
| Ex. 3 | 21 | 5.5 | 13.2 |
| | 22 | 5.3 | 13.5 |
| | 40 | 5.6 | 13.0 |
| | 69 | 5.4 | 13.4 |
| Comp. Ex. 1 | NPB | 6 | 12.4 |

As seen from Table 3, the compounds of the present invention exhibit better performance than the existing material.

### Example 4

### Manufacture of OLED Device Using the Compound for Organic Electronic Material According to the Present Invention

An ITO substrate was mounted on a substrate holder of a vacuum deposition apparatus in the same manner as Comparative Example 1. Then, after adding Compound 40 in a cell of the vacuum deposition apparatus, the pressure inside the chamber was reduced to 10⁻⁶ torr. Then, Compound 40 was evaporated by applying electrical current to the cell to form a hole injection layer having a thickness of 60 nm on the ITO substrate.

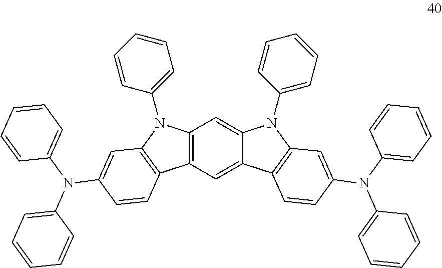

Subsequently, after adding N,N′-bis(α-naphthyl)-N,N′-diphenyl-4,4′-diamine (NPB) in another cell of the vacuum deposition apparatus, NPB was evaporated by applying electrical current to the cell to form a hole transport layer having a thickness of 20 nm on the hole injection layer.

An OLED device was manufactured with other conditions the same as those of Comparative Example 1.

Luminous efficiency of the OLED devices manufactured in Example 4 and Comparative Example 1 was measured at 1,000 cd/m². The result is given in Table 4.

TABLE 4

| | | | Luminous |
|---|---|---|---|
| | Hole | | efficiency |
| | injection | Driving voltage | (cd/A) @ |
| No. | material | (V) @ 1,000 cd/m² | 1,000 cd/m² |
| Ex. 4 | 40 | 5.2 | 13.0 |
| Comp. Ex. 1 | 2-TNATA | 6 | 12.4 |

As seen from Table 4, the compound of the present invention exhibits better performance than the existing material.

### Example 5

### Manufacture of OLED Device Using the Compound for Organic Electronic Material According to the Present Invention

A hole injection layer and a hole transport layer were formed in the same manner as Example 1. Subsequently, after adding dinaphthylanthracene (DNA) in a cell of the vacuum deposition apparatus as an electroluminescent host material and adding DSA-Ph in another cell as in Example 1, an electroluminescent layer was formed on the hole transport layer at a deposition rate of 100:3.

Subsequently, after depositing the compound according to the present invention (e.g. Compound 42) with a thickness of 20 nm as an electron transport layer, lithium quinolate (Liq) was deposited thereon with a thickness of 1 to 2 nm as an electron injection layer. Then, an Al cathode having a thickness of 150 nm was formed using another vacuum deposition apparatus to manufacture an OLED.

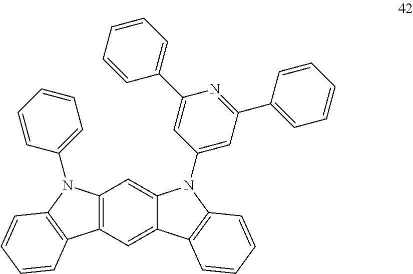

Luminous efficiency of the OLED devices manufactured in Example 5 and Comparative Example 1 was measured at 1,000 cd/m². The result is given in Table 5.

TABLE 5

| | | | Luminous |
|---|---|---|---|
| | Electron | | efficiency |
| | transport | Driving voltage | (cd/A) @ |
| No. | material | (V) @ 1,000 cd/m² | 1,000 cd/m² |
| Ex. 5 | 42 | 5.1 | 12.6 |
| | 43 | 5.2 | 12.7 |
| | 44 | 5.1 | 12.5 |
| | 45 | 5.2 | 12.7 |
| | 46 | 5.1 | 12.6 |
| Comp. Ex. 1 | Alq | 6 | 12.4 |

As seen from Table 5, the compound of the present invention exhibits better performance than the existing material.

### Example 6

### Manufacture of OLED Device Using the Compound for Organic Electronic Material According to the Present Invention

A hole injection layer and a hole transport layer were formed in the same manner as Example 1. Subsequently, after adding Compound 47 in a cell of the vacuum deposition apparatus as a phosphorescent host and adding Ir(ppy)₃ in another cell as a green-emitting dopant, the two materials were evaporated at different rate such that an electroluminescent layer having a thickness of 30 nm was formed on the hole transport layer. Preferred doping concentration was 4 to 10 wt % based on the host.

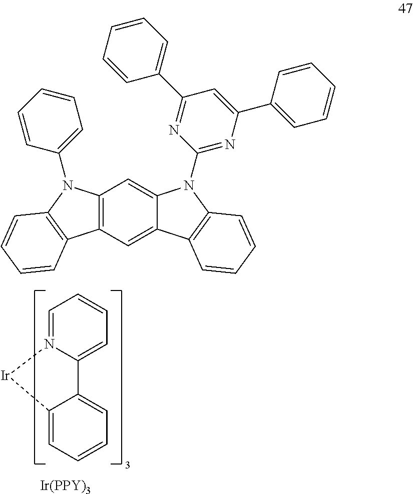

Subsequently, an electron transport layer and an electron injection layer were formed in the same manner as Example 1 and an Al cathode having a thickness of 150 nm was formed using another vacuum deposition apparatus to manufacture an OLED.

### Comparative Example 2

### Electroluminescent Property of OLED Device Using Existing Electroluminescent Material

A hole injection layer and a hole transport layer were formed in the same manner as Example 1. Then, after adding 4,4′-N,N′-dicarbazole-biphenyl (CBP) in a cell of the vacuum deposition apparatus as an electroluminescent host material and adding Ir(ppy)₃ in another cell as a green-emitting dopant, the two materials were evaporated at different rate such that an electroluminescent layer having a thickness of 30 nm was formed on the hole transport layer. Preferred doping concentration was 4 to 10 wt % based on the host.

Subsequently, after depositing bis(2-methyl-8-quinolinato)(p-phenylphenolato)aluminum(III) (BAlq) on the electroluminescent layer with a thickness of 5 nm as a hole blocking layer, an electron transport layer and an electron injection layer were formed in the same manner as Example 1 and an Al cathode having a thickness of 150 nm was formed using another vacuum deposition apparatus to manufacture an OLED.

Driving voltage and green luminous efficiency of the OLED devices manufactured in Example 6 and Comparative Example 2 were measured at 10 mA/cm². The result is given in Table 6.

TABLE 6

| | | | | | Maximum | | |
|---|---|---|---|---|---|---|---|
| | | | Hole | Driving | luminous | | |
| | | | blocking | voltage | efficiency | Color | Emitted |
| No. | Host | Dopant | layer | (V) | (cd/A) | coordinates | color |
| Ex. 6 | 47 | Ir(ppy)₃ | — | 6.6 | 26.5 | (0.281, 0.606) | Green |
| | 48 | Ir(ppy)₃ | — | 6.5 | 26.7 | (0.281, 0.607) | Green |
| | 53 | Ir(ppy)₃ | — | 6.5 | 26.4 | (0.281, 0.607) | Green |
| | 58 | Ir(ppy)₃ | BAlq | 6.7 | 26.3 | (0.281, 0.606) | Green |
| | 59 | Ir(ppy)₃ | BAlq | 6.8 | 26.4 | (0.281, 0.606) | Green |
| Comp. | CBP | Ir(ppy)₃ | BAlq | 7.5 | 25.1 | (0.302, 0.604) | Green |
| Ex. 2 | | | | | | | |

When compared with the existing electroluminescent host CBP, the devices wherein the compounds according to the present invention were used as phosphorescent host exhibited no change in the main EL peak but significantly smaller x value in the color coordinates because of decreased FWHM. Further, the driving voltage was lower than the device wherein CBP was used as host by 0.6 V or more. Accordingly, it can be seen that, when used as a green phosphorescent host, the compounds according to the present invention can significantly reduce power consumption as compared to the existing material and that the process of device manufacture may be simplified because good luminous efficiency is attained even without a hole blocking layer.

The present application contains subject matter related to Korean Patent Application No. 10-2009-0027221, filed in the Korean Intellectual Property Office on Mar. 31, 2009, the entire contents of which is incorporated herein by reference.

While the present invention has been described with respect to the specific embodiments, it will be apparent to those skilled in the art that various changes and modifications may be made without departing from the spirit and scope of the invention as defined in the following claims.

1. A compound for an organic electronic material represented by Chemical Formula 1: 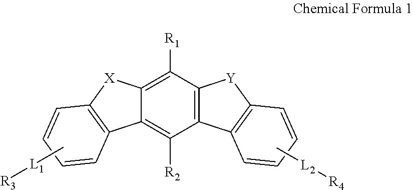
wherein
X and Y independently represent —C(R₅₁)(R₅₂)—, —N(R₅₃)—, —S—, —O—, —Si(R₅₄)(R₅₅)—, —P(R₅₆)—, —P(═O)(R₅₇)—, —C(═O)— or —B(R₅₈)—;
R₁ through R₄ and R₅₁ through R₅₈ independently represent hydrogen, deuterium, halogen, (C1-C30)alkyl with or without substituent(s), (C6-C30)aryl with or without substituent(s), (C6-C30)aryl with or without substituent(s) fused with one or more (C3-C30)cycloalkyl(s) with or without substituent(s), (C3-C30)heteroaryl with or without substituent(s), 5- to 7-membered heterocycloalkyl with or without substituent(s), 5- to 7-membered heterocycloalkyl fused with one or more aromatic ring(s) with or without substituent(s), (C3-C30)cycloalkyl with or without substituent(s), (C3-C30)cycloalkyl fused with one or more aromatic ring(s) with or without substituent(s), adamantyl with or without substituent(s), (C7-C30)bicycloalkyl with or without substituent(s), cyano, NR₂₁R₂₂, BR₂₃R₂₄, PR₂₅R₂₆, P(═O)R₂₇R₂₈ [wherein R₂₁ through R₂₈ independently represent (C1-C30)alkyl with or without substituent(s), (C6-C30)aryl with or without substituent(s), or (C3-C30)heteroaryl with or without substituent(s).], tri(C1-C30)alkylsilyl with or without substituent(s), di(C1-C30)alkyl(C6-C30)arylsilyl with or without substituent(s), tri(C6-C30)arylsilyl with or without substituent(s), (C6-C30)ar(C1-C30)alkyl with or without substituent(s), (C1-C30)alkyloxy with or without substituent(s), (C1-C30)alkylthio with or without substituent(s), (C6-C30)aryloxy with or without substituent(s), (C6-C30)arylthio with or without substituent(s), (C1-C30)alkoxycarbonyl with or without substituent(s), (C1-C30)alkylcarbonyl with or without substituent(s), (C6-C30)arylcarbonyl with or without substituent(s), (C2-C30)alkenyl with or without substituent(s), (C2-C30)alkynyl with or without substituent(s), (C6-C30)aryloxycarbonyl with or without substituent(s), (C1-C30)alkoxycarbonyloxy with or without substituent(s), (C1-C30)alkylcarbonyloxy with or without substituent(s), (C6-C30)arylcarbonyloxy with or without substituent(s), (C6-C30)aryloxycarbonyloxy with or without substituent(s), carboxyl, nitro, hydroxyl, 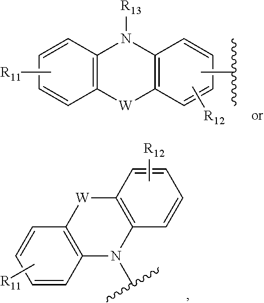
or each of them may be linked to an adjacent substituent via (C3-C30)alkylene or (C3-C30)alkenylene with or without a fused ring to form an aliphatic ring or a monocyclic or polycyclic aromatic ring;
R₁₁ through R₁₃ are the same as defined in R₁ through R₄;
W represents —C(R₅₁R₅₂)ₘ—, —N(R₅₃)—, —S—, —O—, —Si(R₅₄)(R₅₅)—, —P(R₅₆)—, —P(═O)(R₅₇)—, —C(═O)—, —B(R₅₈)— or —(R₅₁)C═C(R₅₂)—;
L₁ and L₂ independently represent a chemical bond, (C6-C30)arylene with or without substituent(s), (C3-C30)heteroarylene with or without substituent(s), 5- or 6-membered heterocycloalkylene with or without substituent(s), 5- to 7-membered heterocycloalkylene fused with one or more aromatic ring(s) with or without substituent(s), (C3-C30)cycloalkylene with or without substituent(s), (C3-C30)cycloalkylene fused with one or more aromatic ring(s) with or without substituent(s), adamantylene with or without substituent(s), (C7-C30)bicycloalkylene with or without substituent(s), (C2-C30)alkenylene with or without substituent(s), (C6-C30)ar(C1-C30)alkylene with or without substituent(s) (C1-C30)alkylenethio with or without substituent(s), (C1-C30)alkyleneoxy with or without substituent(s), (C6-C30)aryleneoxy with or without substituent(s), (C6-C30)arylenethio with or without substituent(s), —O—, —S—, 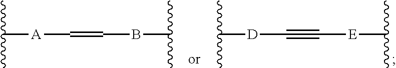
A, B, D and E independently represent a chemical bond, (C6-C30)arylene with or without substituent(s) or (C3-C30)heteroarylene with or without substituent(s);
the heterocycloalkyl or the heteroaryl may contain one or more heteroatom(s) selected from B, N, O, S, P(═O), Si and P; and
m represents an integer 1 or 2.

2. The compound for an organic electronic material according to claim 1, wherein the substituent of R₁ through R₄, R₁₁ through R₁₃, R₂₁ through R₂₈, R₅₁ through R₅₈, L₁, L₂, A, B, D and E is further substituted by one or more substituent(s) selected from a group consisting of deuterium, halogen, (C1-C30)alkyl with or without halogen substituent(s), (C6-C30)aryl, (C3-C30)heteroaryl with or without (C6-C30)aryl substituent(s), 5- to 7-membered heterocycloalkyl, 5- to 7-membered heterocycloalkyl fused with one or more aromatic ring(s), (C3-C30)cycloalkyl, (C3-C30)cycloalkyl fused with one or more aromatic ring(s), tri(C1-C30)alkylsilyl, di(C1-C30)alkyl(C6-C30)arylsilyl, tri(C6-C30)arylsilyl, adamantyl, (C7-C30)bicycloalkyl, (C2-C30)alkenyl, (C2-C30)alkynyl, cyano, carbazolyl, NR₃₁R₃₂, BR₃₃R₃₄, PR₃₅R₃₆, P(═O)R₃₇R₃₈ [wherein R₃₁ through R₃₈ independently represent (C1-C30)alkyl with or without substituent(s), (C6-C30)aryl with or without substituent(s) or (C3-C30)heteroaryl with or without substituent(s)], (C6-C30)ar(C1-C30)alkyl, (C1-C30)alkyl(C6-C30)aryl, (C1-C30)alkyloxy, (C1-C30)alkylthio, (C6-C30)aryloxy, (C6-C30)arylthio, (C1-C30)alkoxycarbonyl, (C1-C30)alkylcarbonyl, (C6-C30)arylcarbonyl, (C6-C30)aryloxycarbonyl, (C1-C30)alkoxycarbonyloxy, (C1-C30)alkylcarbonyloxy, (C6-C30)arylcarbonyloxy, (C6-C30)aryloxycarbonyloxy, carboxyl, nitro and hydroxyl, or is linked to an adjacent substituent to form a ring.

3. The compound for an organic electronic material according to claim 1, wherein 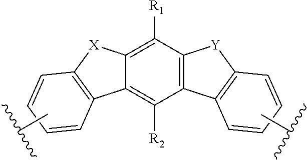
is selected from the following structures: 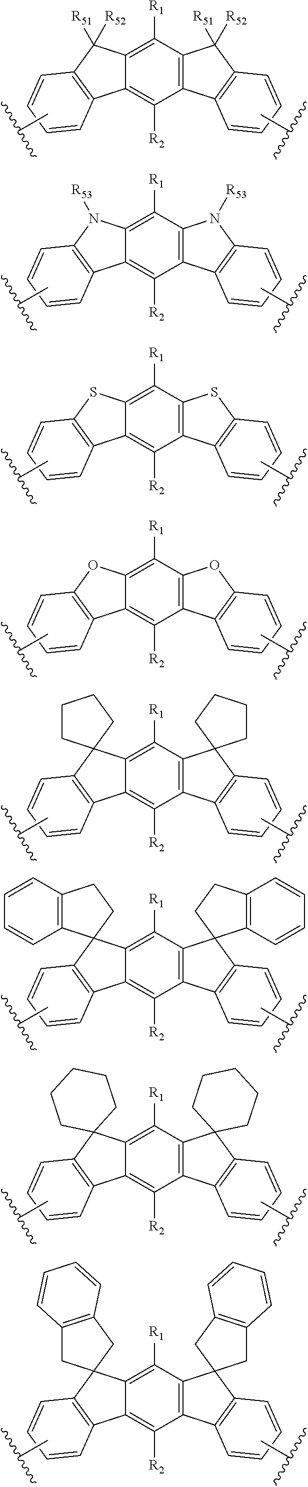 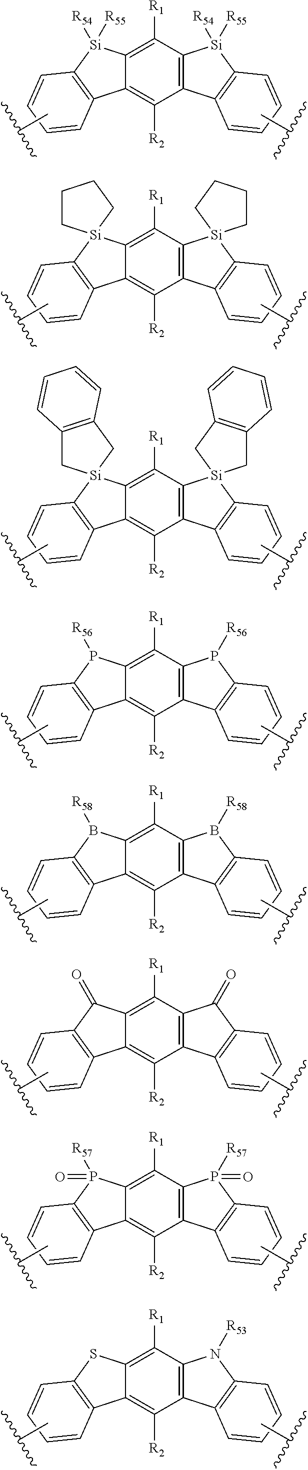 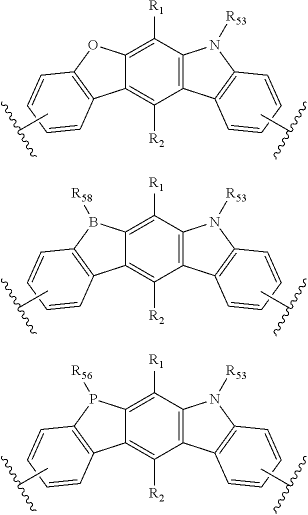
wherein R₁, R₂ and R₅₁ through R₅₈ are the same as defined in claim 1.

4. An organic electronic device comprising the compound for an organic electronic material according to any of claims 1 to 3.

5. The organic electronic device according to claim 4, which comprises a first electrode; a second electrode; and at least one organic layer(s) interposed between the first electrode and the second electrode, wherein the organic layer comprises one or more of the compound(s) for an organic electronic material according to any of claims 1 to 3, and one or more dopant(s) selected from the compounds represented by Chemical Formulas 2 through 4, or one or more host(s) selected from the compounds represented by Chemical Formula 5 or 6: 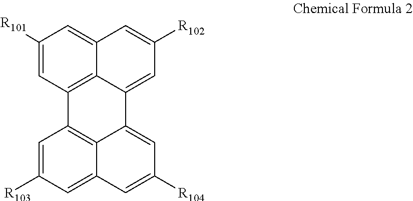
R₁₀₁ through R₁₀₄ independently represent hydrogen, halogen, (C1-C30)alkyl with or without substituent(s), (C6-C30)aryl with or without substituent(s), (C4-C30)heteroaryl with or without substituent(s), 5- or 6-membered heterocycloalkyl with or without substituent(s), 5- to 7-membered heterocycloalkyl fused with one or more aromatic ring(s) with or without substituent(s), (C3-C30)cycloalkyl with or without substituent(s), (C3-C30)cycloalkyl fused with one or more aromatic ring(s) with or without substituent(s), adamantyl with or without substituent(s), (C7-C30)bicycloalkyl with or without substituent(s), cyano, NR₁₁R₁₂, BR₁₃R₁₄, PR₁₅R₁₆, P(═O)R₁₇R₁₈ [wherein R₁₁ through R₁₈ independently represent (C1-C30)alkyl with or without substituent(s), (C6-C30)aryl with or without substituent(s), or (C3-C30)heteroaryl with or without substituent(s).], tri(C1-C30)alkylsilyl with or without substituent(s), di(C1-C30)alkyl(C6-C30)arylsilyl with or without substituent(s), tri(C6-C30)arylsilyl with or without substituent(s), (C6-C30)ar(C1-C30)alkyl with or without substituent(s), (C1-C30)alkyloxy with or without substituent(s), (C1-C30)alkylthio with or without substituent(s), (C6-C30)aryloxy with or without substituent(s), (C6-C30)arylthio with or without substituent(s), (C1-C30)alkoxycarbonyl with or without substituent(s), (C1-C30)alkylcarbonyl with or without substituent(s), (C6-C30)arylcarbonyl with or without substituent(s), (C2-C30)alkenyl with or without substituent(s), (C2-C30)alkynyl with or without substituent(s), (C6-C30)aryloxycarbonyl with or without substituent(s), (C1-C30)alkoxycarbonyloxy with or without substituent(s), (C1-C30)alkylcarbonyloxy with or without substituent(s), (C6-C30)arylcarbonyloxy with or without substituent(s), (C6-C30)aryloxycarbonyloxy with or without substituent(s), carboxyl, nitro or hydroxyl, or each of them may be linked to an adjacent carbon via (C3-C30)alkylene or (C3-C30)alkenylene with or without a fused ring to form a fused ring; 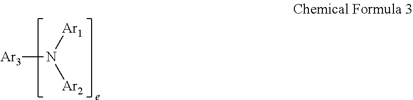
wherein
Ar₁ and Ar₂ independently represent (C1-C30)alkyl with or without substituent(s), (C6-C30)aryl with or without substituent(s), (C4-C30)heteroaryl with or without substituent(s), (C6-C30)arylamino with or without substituent(s), (C1-C30)alkylamino with or without substituent(s), 5- to 7-membered heterocycloalkyl with or without substituent(s), 5- to 7-membered heterocycloalkyl fused with one or more aromatic ring(s) with or without substituent(s), (C3-C30)cycloalkyl with or without substituent(s), or (C3-C30)cycloalkyl fused with one or more aromatic ring(s) with or without substituent(s), or Ar₁ and Ar₂ are linked via (C3-C30)alkylene or (C3-C30)alkenylene with or without a fused ring to form an aliphatic ring or a monocyclic or polycyclic aromatic ring;
in case e is 1, Ar₃ is (C6-C30)aryl with or without substituent(s), (C4-C30)heteroaryl with or without substituent(s) or a substituent selected form the following structures: 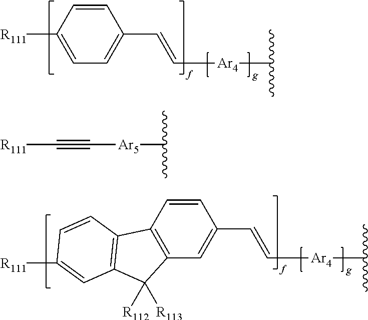
in case e is 2, Ar₃ is (C6-C30)arylene with or without substituent(s), (C4-C30)heteroarylene with or without substituent(s) or a substituent selected form the following structures: 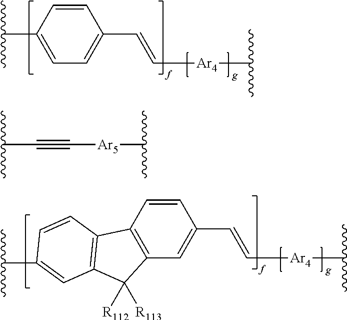
Ar₄ and Ar₅ independently represent (C6-C30)arylene with or without substituent(s) or (C4-C30)heteroarylene with or without substituent(s);
R₁₁₁ through R₁₁₃ independently represent hydrogen, deuterium, (C1-C30)alkyl with or without substituent(s) or (C6-C30)aryl with or without substituent(s);
f is an integer from 1 to 4; and
g is an integer 0 or 1;
M¹L¹⁰¹L¹⁰²L¹⁰³ Chemical Formula 4
wherein
M¹ is selected from a group consisting of Group 7, Group 8, Group 9, Group 10, Group 11, Group 13, Group 14, Group 15 and Group 16 metals;
the ligands L¹⁰¹, L¹⁰² and L¹⁰³ are independently selected from the following structures: 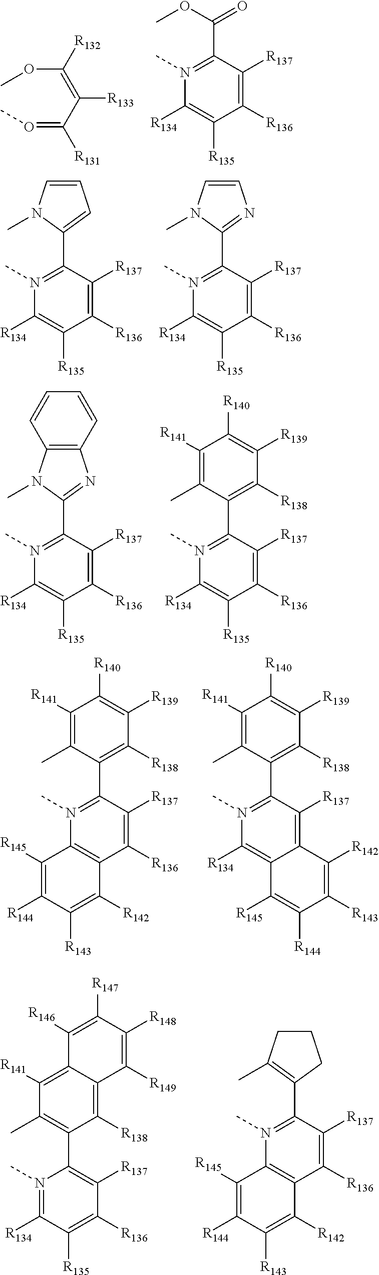 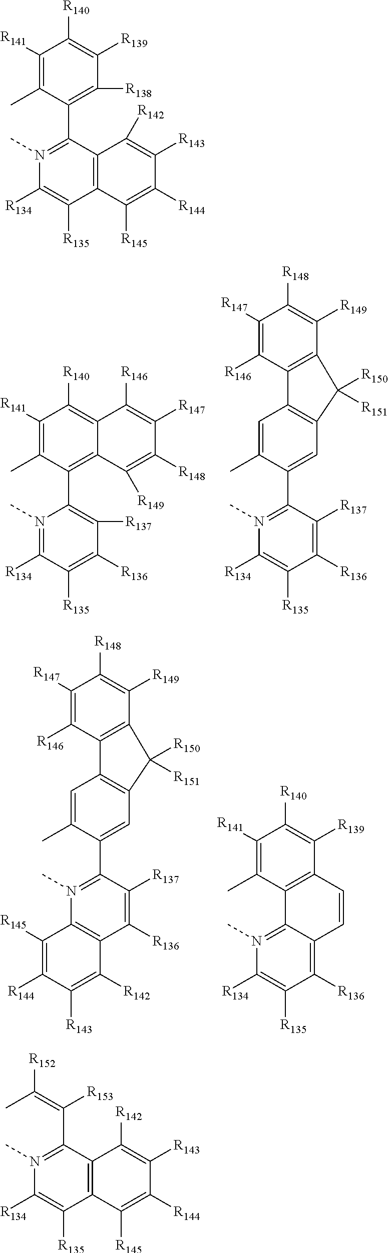 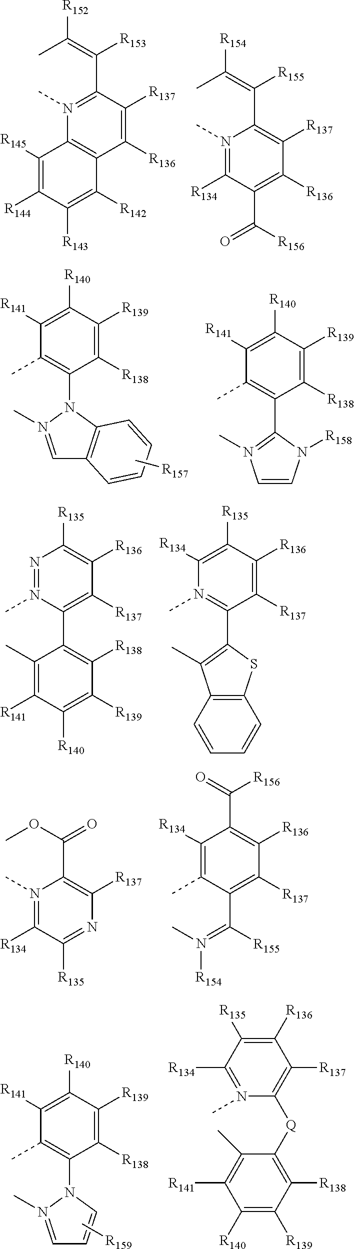
wherein
R₁₃₁ through R₁₃₃ independently represent hydrogen, (C1-C30)alkyl substituted or unsubstituted by halogen(s), (C6-C30)aryl substituted or unsubstituted by (C1-C30)alkyl or halogen;
R₁₃₄ through R₁₄₉ independently represent hydrogen, (C1-C30)alkyl with or without substituent(s), (C1-C30)alkoxy with or without substituent(s), (C3-C30)cycloalkyl with or without substituent(s), (C2-C30)alkenyl with or without substituent(s), (C6-C30)aryl with or without substituent(s), (C1-C30)alkylamino with or without substituent(s), (C6-C30)arylamino with or without substituent(s), SF₅, tri(C1-C30)alkylsilyl with or without substituent(s), di(C1-C30)alkyl(C6-C30)arylsilyl with or without substituent(s), tri(C6-C30)arylsilyl with or without substituent(s), cyano or halogen;
R₁₅₀ through R₁₅₃ independently represent hydrogen, (C1-C30)alkyl substituted or unsubstituted by halogen or (C6-C30)aryl substituted or unsubstituted by (C1-C30)alkyl;
R₁₅₄ and R₁₅₅ independently represent hydrogen, (C1-C30)alkyl with or without substituent(s), (C6-C30)aryl with or without substituent(s) or halogen, or R₁₅₄ and R₁₅₅ are linked via (C3-C12)alkylene or (C3-C12)alkenylene with or without a fused ring to form an aliphatic ring or a monocyclic or polycyclic aromatic ring;
R₁₅₆ represents (C1-C30)alkyl with or without substituent(s), (C6-C30)aryl with or without substituent(s), (C5-C30)heteroaryl with or without substituent(s) or halogen;
R₁₅₇ through R₁₅₉ independently represent hydrogen, (C1-C30)alkyl with or without substituent(s), (C6-C30)aryl with or without substituent(s) or halogen;
Q represents 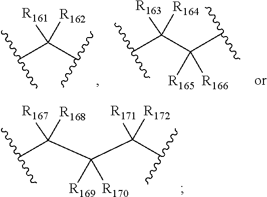
and
R₁₆₁ through R₁₇₂ independently represent hydrogen, (C1-C30)alkyl substituted or unsubstituted by halogen, (C1-C30)alkoxy, halogen, (C6-C30)aryl with or without substituent(s), cyano or (C5-C30)cycloalkyl with or without substituent(s), or each of them may be linked to an adjacent substituent via alkylene or alkenylene to form a spiro-ring or a fused ring, or each of them may be linked with R₁₃₇ or R₁₃₈ via alkylene or alkenylene to form a fused ring; and
(Ar₁₁)ₕ-L₁₁-(Ar₁₂)ᵢ Chemical Formula 5
(Ar₁₃)ⱼ-L₁₂-(Ar₁₄)ₖ [Chemical Formula 6]
wherein
L₁₁ represents (C6-C30)arylene with or without substituent(s) or (C4-C30)heteroarylene with or without substituent(s);
L₁₂ represents anthracenylene with or without substituent(s);
Ar₁₁ through Ar₁₄ are independently selected from hydrogen, (C1-C30)alkyl with or without substituent(s), (C1-C30)alkoxy with or without substituent(s), halogen, (C4-C30)heteroaryl with or without substituent(s), (C5-C30)cycloalkyl with or without substituent(s) and (C6-C30)aryl with or without substituent(s); and
h, i, j and k are independently an integer from 0 to 4.

6. The organic electronic device according to claim 5, wherein the organic layer comprises one or more compound(s) selected from a group consisting of arylamine compounds and styrylarylamine compounds.

7. The organic electronic device according to claim 5, wherein the organic layer further comprises one or more metal(s) or complex(es) selected from a group consisting of organic metals of Group 1, Group 2, 4th period and 5th period transition metals, lanthanide metals and d-transition elements.

8. The organic electronic device according to claim 5, wherein the organic layer comprises an electroluminescent layer and a charge generating layer.

9. The organic electronic device according to claim 5, which is a white light-emitting organic electroluminescent device wherein the organic layer comprises one or more organic electroluminescent layer(s) emitting blue, red or green light.
